SEQUENCE LISTING (1 sequence)

SEQ ID NO: 1 - protein, 191 aa, Homo sapiens [sequence omitted]

(19) United States
(12) Patent Application Publication
(10) Pub. No.: US 2025/0325560 A1
(43) Pub. Date: Oct. 23, 2025

(54) METHODS FOR IMPROVING MUSCLE MASS, STRENGTH, OR FUNCTION WITH A COMBINATION OF TESTOSTERONE AND GROWTH HORMONE

(71) Applicant: University of Rochester, Rochester, NY (US)

(72) Inventor: Chad R. Heatwole, Rochester, NY (US)

(73) Assignee: University of Rochester, Rochester, NY (US)

(21) Appl. No.: 18/870,086

(22) PCT Filed: Jul. 18, 2023
(86) PCT No.: PCT/US2023/070402

§ 371 Date: Nov. 27, 2024

Related U.S. Application Data
(60) Provisional application No. 63/369,867, filed on Jul. 29, 2022.

(51) Int. Cl. A61K 31/568; A61K 38/27; A61P 21/06

(52) U.S. Cl. CPC A61K 31/568; A61K 38/27; A61P 21/06

(57) ABSTRACT

The present disclosure relates to methods, uses, compositions, and kits for improving muscle mass, muscle strength, or muscle function or for reducing fatigue, pain, or obesity in a subject using testosterone or testosterone derivative and growth hormone (GH) or GH derivative. One aspect of this disclosure relates to a combination therapy of testosterone or testosterone derivative and GH or GH derivative for treating various disorders associated with muscle wasting or conditions associated with muscle weakness.

### CROSS REFERENCE TO RELATED APPLICATION

This application claims priority to U.S. Provisional Application No. 63/369,867 filed on Jul. 29, 2022. The content of the application is incorporated herein by reference in its entirety.

### GOVERNMENT INTERESTS

This invention was made with government support under NS095813 awarded by the National Institutes of Health. The government has certain rights in the invention.

### REFERENCE TO SEQUENCE LISTING

The present application is being filed along with a Sequence Listing in electronic format. The Sequence Listing is provided as a file entitled SeqList_161118-03601, created on Jul. 12, 2023, which is 2,078 bytes in size. The information in the electronic format of the sequence listing is incorporated herein by reference in its entirety.

### FIELD OF THE INVENTION

This disclosure relates to methods or uses for improving muscle mass, strength, and function, or methods or uses for treating disorders associated with muscle wasting or muscle weakness, or methods or uses for reducing fatigue, pain, or obesity.

### BACKGROUND

Muscles, the largest tissues in the human body, are essential for various body functions, such as movement, support, protection, heat generation, and blood circulation. Many disorders, injuries, and conditions affect muscle mass, strength, and function. Muscle wasting is a loss of muscle mass due to the muscles weakening and shrinking while muscle weakness is characterized by a lack of strength in the muscles. There are various possible causes of muscle wasting and/or muscle weakness, including certain medical conditions, such as muscular dystrophy and amyotrophic lateral sclerosis. Deficits in muscle function have serious impacts on quality of life. There is a need for improving muscle mass, strength, and function and for treating disorders associated with muscle wasting or muscle weakness.

### SUMMARY

This disclosure addresses the need mentioned above in a number of aspects.

In one aspect, the disclosure provides a method for (i) improving muscle mass, muscle strength, or muscle function, (ii) treating disorders associated with muscle wasting or muscle weakness, or (iii) reducing fatigue, pain, or obesity in a subject in need thereof. The method comprises administering to the subject an effective amount of testosterone or a derivative thereof, and administering to the subject an effective amount of growth hormone (GH) or a derivative thereof.

The disclosure also provides use of (A) an effective amount of testosterone or a derivative thereof, or (B) an effective amount of growth hormone or a derivative thereof, or (C) both in the manufacture of a medicament for (i) improving muscle mass, muscle strength, or muscle function, (ii) treating a disorder associated with muscle wasting or muscle weakness, or (iii) reducing fatigue, pain, or obesity. The improving, treating or reducing comprises administering to a subject in need thereof an effective amount of testosterone or a derivative thereof, and administering to the subject an effective amount of growth hormone or a derivative thereof.

The subject can be a healthy subject, or may have a disorder or condition associated with muscle wasting or muscle weakness, or is at risk of developing the disorder or condition.

Examples of the disorder or condition include Facioscapulohumeral muscular dystrophy (FSHD), Sarcopenia, Duchenne Muscular dystrophy, Limb Girdle Muscular dystrophy, Becker's Muscular dystrophy, Pompe disease, Myotonic dystrophy type-1, myotonic dystrophy type-2, inclusion body myositis, polymyositis, dermatomyositis, amyotrophic lateral sclerosis (ALS), spinal muscular atrophy, Charcot Marie Tooth disease, HIV myopathy, wasting of the elderly, deconditioning, nutritional deficiency, injury, wasting associated with cancer, muscle dysfunction, nerve dysfunction, neuromuscular junction dysfunction, and motor neuron disease. In one example, the disorder is FSHD.

Various testosterone derivatives can be used. In one embodiment, the derivative is a testosterone ester. In some embodiments, the derivative is one having the structure of Formula (II):

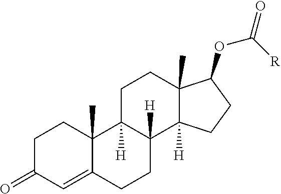

wherein R is alkyl, alkanediyl, alkenyl, alkenediyl, alknyl, aralkyl, aryl, heteroaryl, or acyl. Examples of the testosterone derivative include testosterone enanthate, testosterone propionate, testosterone cypionate, testosterone undecanoate, testosterone oleate, and testosterone palmitate. In one example, the derivative is testosterone enanthate. The testosterone or derivative thereof can be administered at about 0.1 mg to 30,000 mg (e.g., 1 to 10,000 mg, 10 to 1000 mg, 20 to 800 mg, 30 to 600 mg, 40 to 500 mg, and 50 to 200 mg). In some embodiments, the testosterone or derivative thereof is administered at about 70 to 170 mg once every two weeks or at about 110 to 150 mg once every two weeks. For instance, the testosterone or derivative thereof may be administered at about 110 mg, 115 mg, 120 mg, 125 mg, 130 mg, 135 mg, 140 mg, 145 mg, or 150 mg once every two weeks. In one example, the testosterone or derivative thereof is administered at about 140 mg once every two weeks. Preferably, the subject is administered with testosterone enanthate at about 140 mg once every two weeks, or is administered with a testosterone derivative at an equivalent daily dose, or weekly dose, or every two week dose. The testosterone or derivative thereof can be administered to the subject via any suitable routes such as intramuscular injection.

The growth hormone or a derivative thereof may be administered at about 0.01 ug/kg/day to 250 μg/kg/day/kg/day (e.g., 0.1 to 200 μg/kg/day, 0.5 to 100 μg/kg/day, 1.0 to 50 μg/kg/day, 1.5 to 20 μg/kg/day, 2.0 to 10 μg/kg/day). In one embodiment, the growth hormone or derivative thereof can be administered at about 2.5 to 6.0 μg/kg/day or about 4.0 to 5.5 μg/kg/day. In others, the growth hormone or derivative thereof can be administered at about 4.0 μg/kg/day, 4.1 μg/kg/day, 4.2 μg/kg/day, 4.3 μg/kg/day, 4.4 μg/kg/day, 4.5 μg/kg/day, 4.6 μg/kg/day, 4.7 μg/kg/day, 4.8 μg/kg/day, 4.9 μg/kg/day, 5.0 μg/kg/day, 5.1 μg/kg/day, 5.2 μg/kg/day, 5.3 μg/kg/day, 5.4 μg/kg/day or 5.5 μg/kg/day. Preferably, the growth hormone or derivative thereof is administered at about 5.0 μg/kg/day. The growth hormone or derivative thereof can be administered via any suitable routes including subcutaneous injection.

For the method or use described above, the testosterone or derivative thereof, or the growth hormone or derivative thereof may be administered for any suitable duration. In some embodiments, the duration is at least 1 week, e.g., 2 weeks to one or more years, 4 to 72 weeks, 8 to 36 weeks, 12 to 30 weeks, or about 24 weeks.

The method or use can further comprise identifying the subject or evaluating the subject using various tests. Examples of the tests include Quantitative Muscle Testing (QMT), Manual Muscle Testing (MMT), FSHD Clinical Outcome Measure (FSHD-COM), FSHD-Health Index (FSHD-HI), Forced Vital Capacity (FVC), Epworth Sleepiness Scale, Fatigue Severity Scale, Dual Energy X-Ray Absorptiometry (DEXA) Lean Body Mass (total and regional), PROMIS-57, Individualized Neuromuscular Quality of Life Questionnaire (INQoL), Beck Depression Inventory (BDI), and six minute walk distance. One or more of these tests and related step of identifying or evaluating can be carried out before, during, or after the treatment or use.

In another aspect, the disclosure also provides a method or use for treating one or more disorders or conditions described herein in a subject in need thereof. In one example, the disorder is FSHD, The method comprises administering to the subject an effective amount of testosterone or a derivative thereof as described above, and administering to the subject an effective amount of growth hormone or a derivative thereof as described above.

The disclosure also provides a kit for (i) improving muscle mass, muscle strength, or muscle function, (ii) treating disorders associated with muscle wasting or muscle weakness, or (iii) reducing fatigue, pain, or obesity. The kit comprises (a) an effective amount of testosterone or a derivative thereof and (b) an effective amount of growth hormone or a derivative thereof.

The details of one or more embodiments of the disclosure are set forth in the description below. Other features, objectives, and advantages of the disclosure will be apparent from the description and from the claims.

### BRIEF DESCRIPTION OF THE DRAWINGS

FIGS. 1A, 1B, 1C, 1D, 1E, 1F, 1G, and 1H show longitudinal effects of testosterone and rHGH on FSHD patients throughout 36-week study period (24-week treatment period+12-week washout period).

FIG. 1A shows increase in lean body mass.

FIG. 1B shows decrease in fat mass.

FIG. 1C shows improvement in six-minute walk distance.

FIG. 1D shows improvement in Facioscapulohumeral Muscular Dystrophy-Composite Outcome Measure (FSHD-COM).

FIG. 1E shows improvement in standardized Quantitative Muscle Testing (QMT).

FIG. 1F shows improvement in Manual Muscle Testing (MMT).

FIG. 1G shows improvement in Facioscapulohumeral Muscular Dystrophy-Health Index (FSHD-HI) disease burdern.

FIG. 1H shows change in Insulin-like Growth Factor-1 (IGF-1) levels.

FIG. 1I shows changes in free testosterone levels.

### DETAILED DESCRIPTION OF THE INVENTION

This disclosure relates to methods, uses, compositions, and kits for improving muscle mass, muscle strength, or muscle function in a subject using testosterone or testosterone derivative and growth hormone (GH) or GH derivative or variant. One aspect of this disclosure relates to a combination therapy of testosterone or testosterone derivative and GH or GH derivative/variant for treating various disorders associated with muscle wasting or conditions associated with muscle weakness.

Certain aspects of this disclosure are based, at least in part, on an unexpected discovery from a clinical trial where a combination of a testosterone derivative and a recombinant human growth hormone (rHGH) was used as a treatment for individuals with muscles wasting, muscle weakness, limitations walking, functional impairment, or impaired quality of life. As disclosed herein, participants of the trial were found to have an increase in numerous strength and functional measurements with no one experiencing a serious adverse event. Accordingly, testosterone or a testosterone derivative can be used in combination with GH or a GH derivative/variant for treating the disorders associated with muscle wasting or muscle weakness, or for improving muscle mass, muscle strength, or muscle function in a subject.

In one example, a clinical study was conducted to evaluate methods and therapies disclosed herein. More specifically, testosterone enanthate (in oil) was administered via intramuscular injections every 2 weeks in combination with rHGH (GENOTROPIN®) by subcutaneous injections after dinner each evening in a population of adult men with FSHD. Testosterone enanthate, was given at a dose of 140 mg every two weeks and GENOTROPIN® was given at a dose of 5.0 μg/kg/day (calculated using the patient's pre-entry weight). Participants were given this treatment for a 24 week followed by a 12-week washout period.

Nineteen participants completed the study with no participants experiencing a serious adverse event. At 24 weeks, it was found that their mean six-minute-walk distance increased by 37.3 meters (p=0.0007), lean body mass improved by 2.2 kg (p<0.0001), and total disease burden (FSHD-HI) decreased by 19% (p=0.04). The participants were also found to have an increase in numerous strength measurements.

This is notable as patients with FSHD have a progressive decline in ambulation, strength, and function overtime. There is a long-felt unmet medical need for a treatment for FSHD and other forms of muscular dystrophy. There currently are no effective or approved therapies for patients with FSHD and few for any muscular dystrophy. Any treatment strategy that can not only reduce decline but generate a gain in function in muscular dystrophy patients is novel and transformative. Furthermore, the above-described combination therapy is safe, well tolerated, and improves function, muscle mass, and disease burden in men with FSHD. As a non-disease specific therapeutic approach, this combination therapy can also be used as a treatment modality for both men and women with all different types of muscular dystrophy and other medical disorders that result in physical impairment or muscle weakness. Prior to our study, testosterone paired with rHGH had never been studied in any muscular dystrophy population. To the inventor's knowledge, this is the first time these specific preparations and dosages have ever been serially testing using rigorous safety and efficacy modeling in any human population. Thus, the disclosure addresses the above described long-felt unmet medical need.

Disorders and Conditions Associated with Muscle Wasting or Muscle Weakness

The combination therapy disclosed herein can be used for treating various disorders associated with muscle wasting or muscle weakness.

As used herein, a disorder associated with muscle wasting and a muscle wasting-associated disorder are used interchangeably to refer to any condition associated with loss of muscle strength, function, or mass. Examples of these conditions include, but are not limited to, sarcopenia, cachexia, AIDS wasting syndrome, muscular dystrophy (including Duchenne muscular dystrophy syndrome, Becker's muscular dystrophy syndrome, Facioscapulohumeral muscular dystrophy, myotonic dystrophy (type 1 and 2), limb girdle muscular dystrophy, Pompe disease), spinal muscular atrophy, neuromuscular diseases, anorexia, motor neuron diseases, diseases of neuromuscular junction, inflammatory myopathies (e.g. inclusion body myositis, polymyositis, deramatomyositis), disease related to neuropathies (e.g. Charcot Marie Tooth disease, and spinal radiculopathies), nutritional deficiencies, wasting due to cancer, other conditions or diseases associated with decreased muscle mass, and other related diseases. These disorders also include chronic or acute “deconditioning,” as may occur from immobilization or inactivity, such as associated with illness or injury, or the rigors of air travel and space travel. Muscle wasting, including muscle atrophy, can also occur as a consequence of denervation, injury, joint immobilization, enforced bed rest (disuse atrophy), glucocorticoid treatment, sepsis, unweighting, cancer and aging. Jagoe et al. 2001 Curr. Opin. Clin. Nutr. Metab. Care 4: 183. In addition there are a variety of rare forms of myopathy (disorders of carbohydrate metabolism, disorders of lipid metabolism, lysosmal myopathies, inclusion body myopathies, distal myopathies, autoimmune inflammatory myopathies etc) that result in severe pain, weakness, fatigue and disability. The combination therapy disclosed herein is particularly suitable for treating Facioscapulohumeral muscular dystrophy.

A condition associated with muscle weakness and a muscle weakness-associated condition are used interchangeably to refer to any condition associated lack of strength or function in any of the muscles in the body. Examples of the condition include the disorders mentioned above and muscle weakness, fatigue, obesity, or pain in healthy subjects or subjects that do not have any of the above listed disorders.

### Facioscapulohumeral Muscular Dystrophy

Facioscapulohumeral muscular dystrophy (FSHD) is the second most common form of adult muscular dystrophy with a prevalence of 1:15,000-1:20,000 (Padberg G W. Facioscapulohmeral disease (thesis). The Netherlands: The University of Leiden, 1982; Flanigan K M FAU—Coffeen, C M, Coffeen C M FAU—Sexton, L, FAU SL, FAU SD, FAU BS, MF L. Genetic characterization of a large, historically significant Utah kindred with facioscapulohumeral dystrophy). The clinical manifestations of FSHD include steady progressive weakness of the face, shoulders, arms, and hip girdle muscles and life altering fatigue, impaired ambulation, respiratory decline, social limitations, and activity impairment related to muscle weakness.

In 2012 a cross-sectional study of 328 FSHD patients was completed to identify the symptoms most important to this population. Six symptomatic themes were identified as having a prevalence of 90% or higher. These FSHD themes included: (1) problems with shoulders or arms; (2) the inability to do activities; (3) fatigue; (4) back, chest, and abdomen weakness; (5) limitations with mobility or walking; and, (6) changed body image due to disease. In addition to being highly prevalent in FSHD, these themes were also identified as having the highest impact on the daily lives of FSHD patients.

In a recent paper of functional impairment in FSHD, patients were reported to have a loss of strength between 1 and 4% per year and a 24% chance of developing a need for a wheelchair over a six year interval. See Statland J M, et al., Muscle Nerve 2014; 49:520-7. Currently, there is no known disease modifying therapy for FSHD that can improve or limit functional decline in ambulation.

As disclosed herein, a combination therapy of (i) a testosterone or a testosterone derivative and (ii) a GH or a GH derivative can be used to treat FSHD or to improve or ameliorate at least one physical parameter of FSHD. For example, the combination therapy can be used in a treatment for impaired walking in FSHD. The combination therapy can be a treatment for weakness in FSHD. The combination therapy can be a treatment for impaired function in FSHD. The combination therapy can be a treatment for disease burden in FSHD. The combination therapy can be a treatment for muscle atrophy in FSHD. The combination therapy can be used to improve quality of life in FSHD.

In some embodiments, the combination therapy can be used in a treatment for patients with other muscular dystrophy, including by not limited to: Duchenne Muscular dystrophy, Limb Girdle Muscular dystrophy, Becker's Muscular dystrophy, Pompe disease, Myotonic dystrophy type-1, myotonic dystrophy type-2, and others. The combination therapy described above can also be a treatment for patients with functional limitations related to their muscles or nerves including by not limited to: inclusion body myositis, polymyositis, dermatomyositis, ALS, spinal muscular atrophy, Charcot Marie Tooth disease, and others. In some embodiments, the combination therapy can be used as a treatment for patients with sarcopenia of any cause, such as HIV myopathy, wasting of the elderly, deconditioning, nutritional deficiency, injury, or wasting associated with cancer. The combination therapy may also have benefit in female populations including those having the conditions or disorders described above.

### Testosterone and Testosterone Derivatives

Testosterone is a naturally occurring androgen that is produced in both men and women. Testosterone promotes protein synthesis and has anabolic effects on both muscle and bone (Shahidi N T. Clin Ther 2001; 23:1355-90). It is commonly utilized for men with hypogonadism and conditions associated with low or no endogenous testosterone (Bhasin S, et al., Best Pract Res Clin Endocrinol Metab 2011; 25:251-70). It is also recommended for men to improve libido and erectile dysfunction (Bhasin S, et al. J Clin Endocrinol Metab 2010; 95:2536-59). In women, testosterone supplementation has been used for muscle atrophy associated with acquired immune deficiency syndrome, inoperable metastatic breast cancer, low libido, sexual dysfunction, muscle wasting, and as a postmenopausal therapy. See, e.g., Margo K, et al., Am Fam Physician 2006; 73:1591-8; Blackman M R, et al. JAMA 2002; 288:2282-92; Choi H H, et al. J Clin Endocrinol Metab 2005; 90:1531-41; Dolan Looby S E, et al. AIDS 2009; 23:951-9; Dolan S, et al. Arch Intern Med 2004; 164:897-904; Miller K, et al. J Clin Endocrinol Metab 1998; 83:2717-25; and Nachtigall L, et al. Gynecol Endocrinol 2011; 27:39-48.

The Endocrine Society currently recommends testosterone in isolation to: (1) increase muscle strength and lean body mass in patients with HIV; and (2) improve bone mineral densities in patients receiving high dosages of glucocorticoids (Bhasin S, et al. J Clin Endocrinol Metab 2010; 95:2536-59). In a prior study, testosterone in isolation has been shown to be safe and increase lean body mass, reduce body fat, and improve basal metabolic rate in a heterogeneous group of adult muscular dystrophy population (including a FSHD participant) with normal baseline testosterone levels. In this study, results were seen after 3 months of treatment and were comparable to the results demonstrated in a group of normal men receiving the same therapy (Welle S, et al., J Clin Endocrinol Metab 1992; 74:332-5). In a second study of 40 patients with myotonic dystrophy, testosterone in isolation was found to be safe, tolerable, and able to improve both creatinine excretion and lean body mass while not statistically improving overall strength (Griggs R C, et al. Neurology 1989; 219-22).

Without wishing to be bound by theory, testosterone is commonly known to persons of ordinary skill in the art and is shown by compound with the Formula (I):

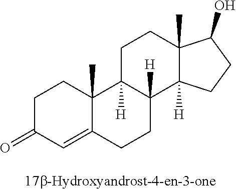

Testosterone is also known under the chemical name 17-β-hydroxyandrost-4-en-3-one (or 4 androsten 17β-ol-3-one) which can be obtained in various ways: it may be isolated and purified from nature or synthetically produced by any manner. As disclosed herein, either testosterone or a testosterone derivative or a testosterone analogue can be used.

As used herein, the terms “derivative,” “variant,” and “analogue” are used interchangeable to refer to a compound having a structure derived from the structure of a parent compound (e.g., a compound disclosed herein, e.g., testosterone or GH) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds.

Exemplary derivatives include salts, esters, amides, salts of esters or amides, and N-oxides of a parent compound. An example is shown in Formula (II), wherein R is alkyl, alkanediyl, alkenyl, alkenediyl, alknyl, aralkyl, aryl, heteroaryl, acyl:

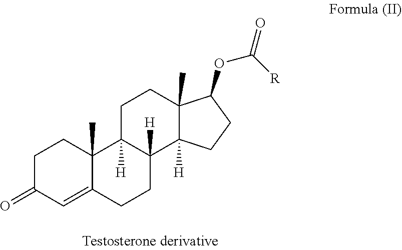

The testosterone derivative or analogue may be prodrug, ester, salt, or metabolite of testosterone. It includes any useful metabolite or precursor of testosterone, for example the metabolite dihydrotestosterone. In some embodiments, a testosterone analogue can be, e.g., a testosterone ester such as testosterone cypionate, enanthate or propionate or a combination thereof, prodrug or fatty acid ester of testosterone; a fatty acid ester of testosterone of long chain (i.e., 14 or more carbons); methyltestosterone (in which the methyl group is covalently bonded to the testosterone nucleus as the C17 position to inhibit hepatic metabolism); a testosterone alkyl ester; an undecanoate acid ester of testosterone; testosterone undecanote; or a composition as disclosed, e.g. in US20200174026 and US20110251167, which are incorporated herein by reference.

A “testosterone ester” as used in this application is a derivative of testosterone comprising at least a substitution on the hydroxyl group on the cyclopentyl ring of the steroid core with an acyl functional group or a substituted acyl functional group as those functional groups are defined below. When a carbon limit is assigned to a testosterone ester, the carbon limit is relative only to the carbon atoms on the acyl substitution.

The term “physiologically cleavable ester” refers to a derivative of the hydroxyl of Formula (II) and an acid or acid derivative, wherein the product is cleaved in the body to give the compound Formula (II) or an active metabolite. Such a physiologically cleavable ester can be viewed as a “prodrug.” Such a “prodrug” is valuable if it increases the bioavailability of the corresponding hydroxyl compound when such a pro-drug is administered to a subject. For example, a “prodrug” administered intranasally may be more readily absorbed into the blood, may facilitate the delivery of the parent compound to a biological compartment of the subject such as the brain or lymphatic, which may also have more favorable patient acceptance, safety profiles and/or pharmacokinetics for specific tailoring to subjects for use in the intended indication. A general overview of pro-drugs is provided in (1) “Pro-drugs As Novel Delivery Systems,” Vol. 14 of the ACS Symposium Series, by T. Higuchi and V. Stella, and (2) “Bioreversible Carriers in Drug Design,” American Pharmaceutical Association, Pergamon Press, 1987, Edward B. Roche, Ed.

Testosterone is esterified in various pharmaceutical preparations, with esters of propionate, enanthate (see Formula (III)), cypionate and undecanoate being marketed as oral or injectable formulations for the treatment of hypogonadism. Formula (III) Testosterone enanthate:

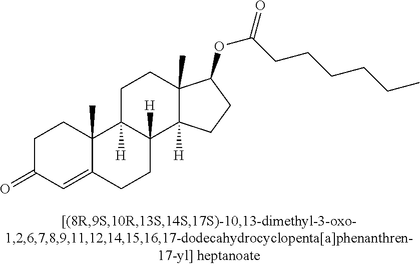

Carboxylic acids that form the “carbonyl group” of the ester, i.e., —C(O)—R, that can be used as derivatives according to the present disclosure and form the “prodrug” include mono-carboxylic acids that are derived from unsubstituted or substituted lower linear or branched chain alkyl, alkenyl, alkynyl or arylakyl entities. R is defined for example below. Naturally occurring carboxylic acids are generally a preferred class of that may as acceptable, cleavable esters of a pharmaceutically-active ingredient.

The term “lower alkyl” carboxylic acid refers to a monovalent, saturated aliphatic hydrocarbon radical having from one to twelve (12) carbon atoms bonded to a carboxyl group. Alkyl may be a straight chain (i.e. linear), a branched chain, or a cyclic structure. Representative examples of lower alkyl radicals include methyl, ethyl, n-propyl, n-butyl, n-pentyl, n-hexyl, isopropyl, isobutyl, isopentyl, amyl, sec-butyl, tert-butyl, tert-pentyl, cyclopropyl, cyclobutyl, cyclopentylethyl (cypionate), undecanoate and the like.

The term “saturated” as used herein means the compound or group so modified has no carbon-carbon double and no carbon-carbon triple bonds, except as noted below. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded.

The term “aliphatic” when used without the “substituted” modifier signifies that the compound/group so modified is an acyclic or cyclic, but non aromatic hydrocarbon compound or group. In aliphatic compounds/groups, the carbon atoms can be joined together in straight chains, branched chains, or non-aromatic rings (alicyclic). Aliphatic compounds/groups can be saturated, that is joined by single bonds (alkanes/alkyl), or unsaturated, with one or more double bonds (alkenes/alkenyl) or with one or more triple bonds (alkynes/alkynyl).

The term “alkyl” when used without the “substituted” modifier refers to a monovalent saturated aliphatic group with a carbon atom as the point of attachment, a linear or branched acyclic structure, and no atoms other than carbon and hydrogen. The groups —CH3 (Me)—CH2CH3 (Et), —CH2CH2CH3 (n-Pr or propyl), —CH(CH3)2 (i-Pr, ^{t}Pr or isopropyl), —CH2CH2CH2CH3 (n-Bu), —CH(CH3)CH2CH3 (sec-butyl), —CH2CH(CH3)2 (isobutyl), —C(CH33 (tert-butyl, t-butyl, t-Bu or ^{t}Bu), and —CH2C(CH33 (neo-pentyl) are non-limiting examples of alkyl groups.

The term “alkanediyl” when used without the “substituted” modifier refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH2- (methylene), —CH2CH2-, —CH2C(CH3) 2CH2-, and —CH2CH2CH2- are non-limiting examples of alkanediyl groups. An “alkane” refers to the compound H—R, wherein R is alkyl as this term is defined above. When any of these terms is used with the “substituted” modifier one or more hydrogen atom has been independently replaced by —OH, —F, —Cl, —Br, —I, —NH2, —NO2, —CO2H, —CO2CH3, —CN, —SH, —OCH3, —OCH2CH3, —C(O)CH3, —NHCH3, —NHCH2CH3, —N(CH3)2, —C(O)NH2, —OC(O)CH3, or S(O)2NH2. The following groups are non-limiting examples of substituted alkyl groups: —CH2OH, —CH2Cl, —CH3, —CH2CN, —CH2C(O)OH, —CH2C(O)CH3, —CH2C(O)NH2, —CH2C(O)CH3, —CH2OCH3, —CH2OC(O)CH3, —CH2NH2, —CH2N(CH3)2, and —CH2CH2Cl.

The term “alkenyl” when used without the “substituted” modifier refers to an monovalent unsaturated aliphatic group with a carbon atom as the point of attachment, a linear or branched acyclic structure, at least one nonaromatic carbon-carbon double bond, no carbon-carbon triple bonds, and no atoms other than carbon and hydrogen. Non-limiting examples include: —CH═CH2 (vinyl), —CH═CHCH3, —CH═CHCH2CH3, —CH2CH═CH2 (allyl), —CH2CH═CHCH3, and —CH═CHCH═CH2.

The term “alkenediyl” when used without the “substituted” modifier refers to a divalent unsaturated aliphatic group, with two carbon atoms as points of attachment, a linear or branched, a linear or branched acyclic structure, at least one nonaromatic carbon-carbon double bond, no carbon-carbon triple bonds, and no atoms other than carbon and hydrogen. The groups —CH═CH—, —CH.dbd.C(CH3)CH2-, —CH═CHCH2-, and —CH2CH═CHCH2- are non-limiting examples of alkenediyl groups. It is noted that while the alkenediyl group is aliphatic, once connected at both ends, this group is not precluded from forming part of an aromatic structure. The terms “alkene” or “olefin” are synonymous and refer to a compound having the formula H R, wherein R is alkenyl as this term is defined above. A “terminal alkene” refers to an alkene having just one carbon-carbon double bond, wherein that bond forms a vinyl group at one end of the molecule. When any of these terms are used with the “substituted” modifier one or more hydrogen atom has been independently replaced by —OH, —F, —Cl, —Br, —I, —NH2, —NO2, —CO2H, —CO2CH3, —CN, —SH, —OCH3, —OCH2CH3, —C(O)CH3, —NHCH3, —NHCH2CH3, —N(CH3) 2, —C(O)NH, —OC(O)CH3, or —S(O) 2NH2. The groups —CH═CHF, —CH═CHCl and —CFI═CHBr are non-limiting examples of substituted alkenyl groups.

The term “alkynyl” when used without the “substituted” modifier refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —CCH, —CCCH3, and —CH2CCCH3 are non-limiting examples of alkynyl groups. An “alkyne” refers to the compound FI—R, wherein R is alkynyl.

When any of these terms are used with the “substituted” modifier one or more hydrogen atom has been independently replaced by —OH, —F, —Cl, —Br, —I, —NH2, —NO2, —CO2H, —CO2CH3, —CN, —SH, —OCH3, —OCH2CH3, —C(O)CH3, —NHCH3, —NHCH2CH3, —N(CH3)2, —C(O)NH, —OC(O)CH3, or —S(O)2NH2.

The term “aryl” when used without the “substituted” modifier refers to a monovalent unsaturated aromatic group with an aromatic carbon atom as the point of attachment, said carbon atom forming part of a one or more six-membered aromatic ring structure, wherein the ring atoms are all carbon, and wherein the group consists of no atoms other than carbon and hydrogen. If more than one ring is present, the rings may be fused or unfused. As used herein, the term does not preclude the presence of one or more alkyl or aralkyl groups (carbon number limitation permitting) attached to the first aromatic ring or any additional aromatic ring present. Non-limiting examples of aryl groups include phenyl (Ph), methylphenyl, (dimethyl)phenyl, —C6H4CH2CH3 (ethylphenyl), naphthyl, and a monovalent group derived from biphenyl. The term “arenediyl” when used without the “substituted” modifier refers to a divalent aromatic group with two aromatic carbon atoms as points of attachment, said carbon atoms forming part of one or more six-membered aromatic ring structure(s) wherein the ring atoms are all carbon, and wherein the monovalent group consists of no atoms other than carbon and hydrogen. As used herein, the term does not preclude the presence of one or more alkyl, aryl or aralkyl groups (carbon number limitation permitting) attached to the first aromatic ring or any additional aromatic ring present. If more than one ring is present, the rings may be fused or unfused. Unfused rings may be connected via one or more of the following: a covalent bond, alkanediyl, or alkenediyl groups (carbon number limitation permitting). Non-limiting examples of arenediyl groups include:

An “arene” refers to the compound H—R, wherein R is aryl as that term is defined above. Benzene and toluene are non-limiting examples of arenes.

When any of these terms are used with the “substituted” modifier one or more hydrogen atom has been independently replaced by —OH, —F, —Cl, —Br, —I, —NH2, —NO2, —CO2H, —CO2CH3, —CN, —SH, —OCHS, —OCH2CH3, —C(O)CH3, —NHCH3, —NHCH2CH3, —NCH3)2, —C(O)NH2, —OC(O)CH3, or —S(O)2NH2.

The term “aralkyl” when used without the “substituted” modifier refers to the monovalent group -alkanediyl-aryl, in which the terms alkanediyl and aryl are each used in a manner consistent with the definitions provided above. Nonlimiting examples are: phenylmethyl (benzyl, Bn) and 2-phenyl-ethyl.

When the term aralkyl is used with the “substituted” modifier one or more hydrogen atom from the alkanediyl and/or the aryl group has been independently replaced by —OH, —F, —Cl, —Br, —I, —NH2, —NO2, —CO2H, —CO2CH3, —CN, —SH, —OCH3, —OCH2CH3, —C(O)CH3, —NHCH3, —NHCH2CH3, —N(CH3) 2, —C(O)NH2, OC(O)CH3, or —S(O)2NH2. Non-limiting examples of substituted aralkyls are: (3-chlorophenyl)-methyl, and 2-chloro-2-phenyl-eth-1-yl.

The term “heteroaryl” when used without the “substituted” modifier refers to a monovalent aromatic group with an aromatic carbon atom or nitrogen atom as the point of attachment, said carbon atom or nitrogen atom forming part of one or more aromatic ring structures wherein at least one of the ring atoms is nitrogen, oxygen or sulfur, and wherein the heteroaryl group consists of no atoms other than carbon, hydrogen, aromatic nitrogen, aromatic oxygen and aromatic sulfur. If more than one ring is present, the rings may be fused or unfused. As used herein, the term does not preclude the presence of one or more alkyl, aryl, and/or aralkyl groups (carbon number limitation permitting) attached to the aromatic ring or aromatic ring system. Non-limiting examples of heteroaryl groups include furanyl, imidazolyl, indolyl, indazolyl (Im), isoxazolyl, methylpyridinyl, oxazolyl, phenylpyridinyl, pyridinyl, pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the compound H—R, wherein R is heteroaryl. Pyridine and quinoline are non-limiting examples of heteroarenes. When these terms are used with the “substituted” modifier one or more hydrogen atom has been independently replaced by —OH, —F, —Cl, —Br, —I, —NH2, —NO2, —CO2H, —CO2CH3, —CN, —SH, —OCH3, —CH2CH3, —C(O)CH3, —NHCH3, —NHCH2CH3, —N(CH3)2, —C(O)NH, —OC(O)CH3, or —S(O)2NH2.

The term “acyl” when used without the “substituted” modifier refers to the group C(O)R, in which R is a hydrogen, alkyl, cycloalkyl, alkenyl, aryl, aralkyl or heteroaryl, as those terms are defined above. The groups, —CHO, —C(O)CH3 (acetyl, Ac), —C(O)CHCH3, —C(O)CH2CHCH3, —C(O)CH(CH3)2, —C(O)CH(CH)2, —C(O)C6H5, —C(O)C6H4CH3, —C(O)CH2C6H5, —C(O)(imidazolyl) are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkane, as defined above, wherein at least one of the hydrogen atoms has been replaced with a —CHO group. When any of these terms are used with the “substituted” modifier one or more hydrogen atom (including a hydrogen atom directly attached to the carbon atom of the carbonyl or thiocarbonyl group, if any) has been independently replaced by —OH, —F, —Cl, —Br, —I, —NH2, —SH, —OCH3, —OCH2CH3, —NHCH3, —NHCH2CH3, —N(CH3)2, —OC(O)CH3, or —S(O)2NH2. The groups, —C(O)CHCH3, —CO2 (carboxyl), —CO2CH3 (methylcarboxyl), —CO2CH2CH3, —C(O)NH2 (carbamoyl), and —CON(CH3)2, are non-limiting examples of substituted acyl groups. The term lower “alkenyl” carboxylic acid refers to an aliphatic group that has 1-12 carbons, may be straight chain, branched chain, and cyclic groups and with no more than 3 double bonds, all of which may be optionally substituted similarly to the alkyl group. Representative examples of lower alkenyl radicals in carboxylic acids include vinyl (ethenyl), allyl (propen-3-yl), 1-buten-4-yl; 2-buten-4-yl, l-penten-5-yl, and the like.

The term “pharmaceutically-acceptable carboxylic acid” means a carboxylic acid moiety that is useful for forming the pharmaceutical formulations and compositions are also physiologically acceptable and generally non-toxic to a subject receiving the moiety.

Various compositions are available for the therapy described herein, e.g. testosterone patches or injections; intramuscular injections, implants, oral tablets of alkylated T (e.g., methyltestosterone), subcutaneous formulations, intranasal formulations, buccal formulations, transdermal formulations such as the topical gels and solutions, or topical patches, and the like.

In some embodiments, the composition can be a solid dosage formulation (e.g., tablet, capsule, granule, powder, sachet, or chewable), solution, gel, suspension, emulsion, shampoo, conditioner, cream, foam, gel, lotion, ointment, transdermal patch, film, tincture, or paste. Further provided herein are methods and uses of the compositions described herein for treating a disease, preventing a disease, treating a condition, and/or preventing a condition.

The formulation of the testosterone or testosterone derivatives and analogues or salts thereof may provide a dose of testosterone adequate to maintain a male subject's serum total testosterone level within the normal male range (approximately 300 to 1000 ng/dL range), based on measures of serum total testosterone. The pharmaceutically effective amount of the testosterone or testosterone derivatives and analogues or salts thereof present in the compositions as disclosed herein may depend on the patient's starting serum total testosterone and the mode of administration.

For oral administration, the compositions can be provided in the form of tablets containing 0.01, 0.05, 0.1, 0.5, 1.0, 2.5, 5.0, 10.0, 15.0, 25.0, 50.0 and 100 milligrams of active ingredient for the symptomatic adjustment of the dosage to the subject to be treated. An effective amount of the drug can be ordinarily supplied at a dosage level of from about 0.0002 mg/kg to about 50 mg/kg of body weight per day. The range may be more particularly from about 0.001 to 7 mg/kg of body weight per day. In particular, testosterone and testosterone derivatives and analogues or salts thereof delivered by intramuscular injections may be provided in injections of 50 to 750 mg every 1 to 4 weeks. In one embodiment, testosterone and testosterone derivatives and analogues or salts thereof are provided by intramuscular injections of 100 to 500 mg every 1 to 4 weeks. In one class of this embodiment, testosterone and testosterone derivatives and analogues or salts thereof are provided by intramuscular injections of 50 to 250 mg every 1 to 4 weeks.

Testosterone and testosterone derivatives and analogues or salts thereof may be provided in gel or cream forms in doses of 20 to 200 mg per day. In one embodiment, testosterone and testosterone derivatives and analogues or salts thereof are provided in a gel at doses of 50 to 100 mg/day, particularly 50 mg/day, 75 mg/day and 100 mg/day.

Transdermal patches can used to deliver testosterone and testosterone derivatives and analogues or salts thereof of 1 to 10 mg per day, particularly, 4 to 6 mg/day.

Testosterone, testosterone derivatives and analogues or salts thereof may also be provided by means of a buccal gel at a dose of 10 mg/day to 100 mg/day. In one embodiment, the dose of testosterone or testosterone derivatives and analogues or salts thereof is a buccal gel is 40 to 80 mg/day. In one class of this embodiment, the dose of testosterone or testosterone derivatives and analogues or salts thereof in a buccal gel is 60 mg/day.

Commercial testosterone therapies are known in the art, e.g. topical testosterone formulations (e.g., ANDROGEN, AXIRON, FIRST-TESTOSTERONE, FIRST-TESTOSTERONE MC, FORTESTA, and TESTIM); transdermal patch formulations (e.g., ANDRODERM); and buccal testosterone formulations (e.g., STRIANT).

Pharmaceutical compositions containing testosterone, testosterone derivatives, testosterone analogues or salts thereof may further comprise a pharmaceutically acceptable carrier. In certain embodiments, the pharmaceutical composition may be formulated (e.g., using the same excipients in the same ratios and/or comprising the same dose strength) or administrated in the same way as commercially available testosterone, testosterone prodrug, or testosterone derivative products, including but not limited to: ANDRODERM, ANDROGEL, ANDROID 10, ANDROID 25, ANDROID 5, AVEED, AXIRON, DELATESTRYL, DEPO-TESTADIOL, DEPO-TESTOSTERONE, DITATE-DS, FORTESTA, JATENZO, METANDREN, METHYLTESTOSTERONE, NATESTO, ORETON, ORETON METHYL, STRIANT, TESTIM, TESTODERM, TESTODERM TTS, TESTOPEL, Testosterone, Testosterone Cypionate, Testosterone Cypionate-Estradiol Cypionate, Testosterone Enanthate, Testosterone Enanthate And Estradiol Valerate, Testosterone Propionate, Testosterone Undecanoate, TESTRED, VIRILON, VOGELXO, XYOSTED (Autoinjector). The FDA-approved labels for each of these products, as available at the website of the FDA, including with respect to their formulation, dosing, and administration.

GH, GH variants, and GH Derivatives

Growth hormone (GH), also known as somatotropin, is a peptide hormone that stimulates growth, cell reproduction, and cell regeneration in humans and other animals. Human Growth Hormone is produced in the pituitary gland of men and women. This hormone is a single polypeptide chain of 191 amino acids and has a molecular weight of approximately 22 kDa. HGH exhibits a multitude of biological effects, including linear growth (somatogenesis), lactation, activation of macrophages, and insulin-like and diabetogenic effects, among others (Chawla, R., et al., Ann. Rev. Med. 34:519-547 (1983); Isaksson, O., et al., Ann. Rev. Physiol., 47:483-499 (1985); Hughes, J. and Friesen, H., Ann. Rev. Physiol., 47:469-482 (1985)). Shown below is the amino acid sequence of a GH polypeptide encoded by human GH gene, which corresponds to Genbank Accession No. AIA66930.1 or AAF23136.1:

(SEQ ID NO: 1)
FPTIPLSRLFDNAMLRAHRLHQLAFDTYQEFEEAYIPKEQKYSFLQNPQT
SLCFSESIPTPSNREETQQKSNLELLRISLLLIQSWLEPVQFLRSVFANS
LVYGASDSNVYDLLKDLEEGIQTLMGRLEDGSPRTGQIFKQTYSKFDTNS
HNDDALLKNYGLLYCFRKDMDKVETFLRIVQCRSVEGSCGF

The structure of HGH is well known (Goeddel, D., et al., Nature 281:544-548 (1979)), and the three-dimensional structure of HGH has been solved by X-ray crystallography (de Vos, A., et al., Science 255:306-312 (1992)). The protein has a compact globular structure, comprising four amphipathic alpha helical bundles, termed A-D beginning from the N-terminus, which are joined by loops. HGH also contains four cysteine residues, which participate in two intramolecular disulfide bonds: one between Cys-53 and Cys-165, forming a large loop in the molecule, and the other between Cys-182 and Cys-189, forming a small loop near the C-terminus. In solution, HGH exists predominantly as a monomer, with a small fraction as dimers and higher molecular weight oligomers. Under certain conditions, HGH can be induced to form larger amounts of dimers, trimers and higher oligomers.

A number of naturally occurring mutants of HGH have been identified. These include HGH-V (Seeburg, DNA 1: 239 (1982); U.S. Pat. Nos. 4,446,235, 4,670,393, and 4,665,180, which are incorporated by reference herein) and a 20-kDa HGH containing a deletion of residues 32-46 of HGH (Kostyo et al., Biochem. Biophys. Acta 925: 314 (1987); Lewis, U., et al., J. Biol. Chem., 253:2679-2687 (1978)). In addition, numerous HGH variants, arising from post-transcriptional, post-translational, secretory, metabolic processing, and other physiological processes, have been reported (Baumann, G., Endocrine Reviews 12: 424 (1991)).

The biological effects of HGH derive from its interaction with specific cellular receptors. The interaction between HGH and extracellular domain of its receptor is among the most well understood hormone-receptor interactions. High-resolution X-ray crystallographic data (Cunningham, B., et al., Science, 254:821-825 (1991)) has shown that HGH has two receptor binding sites and binds two receptor molecules sequentially using distinct sites on the molecule. The two receptor binding sites are referred to as Site I and Site II. Site I includes the carboxy terminal end of helix D and parts of helix A and the A-B loop, whereas Site II encompasses the amino terminal region of helix A and a portion of helix C. Binding of GH to its receptor occurs sequentially, with Site I binding first. Site II then engages a second GH receptor, resulting in receptor dimerization and activation of the intracellular signaling pathways that lead to cellular responses to the hormone. An HGH mutant in which a G120R substitution has been introduced into site II is able to bind a single HGH receptor, but is unable to dimerize two receptors. The mutant acts as an HGH antagonist in vitro, presumably by occupying receptor sites without activating intracellular signaling pathways (Fuh, G., et al., Science 256:1677-1680 (1992)).

Like testosterone, HGH can stimulate cell growth and regeneration (Giannoulis M G, et al., Endocr Rev 2012; 33:314-77). In HGH deficient states, patients experience exercise intolerance, low bone mineral density, changes in body composition, and a worsening cholesterol profile (Toogood A A, et al., J Clin Endocrinol Metab 1999; 84:131-6). HGH supplementation can ameliorate these impairments. Recombinant HGH (rHGH), a synthesized preparation of HGH, has been used to treat children and adults with growth hormone deficiency, as well as those with muscle wasting, Turner syndrome, Prader-Willi syndrome, chronic renal failure, and idiopathic short stature. See, e.g., Giannoulis M G, et al., Endocr Rev 2012; 33:314-77; Ross J L, et al. N Engl J Med 2011; 364:1230-42; Mogul H R, et al. J Clin Endocrinol Metab 2008; 93:1238-45; Youssef D M. Saudi J Kidney Dis Transpl 2012; 23:755-64; and Kemp S F, et al. J Clin Endocrinol Metab 2005; 90:5247-53.

As disclosed herein, it was unexpectedly found that a combination therapy of GH and testosterone/testosterone derivative led to improvement in muscle mass and strength as well as functional recovery in subjects having disorders associated with muscle wasting or muscle weakness such as FSHD. Accordingly, GH and its derivatives or analogs can be used as therapeutic agents in the therapies disclosed herein. Examples of GH that can be used as therapeutic agents can include GH analogs, GH isoforms, GH mimetics, GH fragments, hybrid GH proteins, fusion proteins oligomers and multimers of the above, homologues of the above, including receptor agonists, glycosylation pattern variants of the above, and mutants of the above, regardless of the method of synthesis or manufacture thereof including but not limited to, recombinant vector expression whether produced from cDNA or genomic DNA, synthetic, transgenic, and gene activated methods.

As used herein, “growth hormone” or “GH” shall include those polypeptides and proteins that have at least one biological activity of a growth hormone from any mammalian species including but not limited to, human (HGH), recombinant human (rHGH), bovine (bGH), porcine, and from other livestock or farm animals including but not limited to, chicken, as well as GH analogs, GH isoforms, GH mimetics, GH fragments, hybrid GH proteins, fusion proteins, oligomers and multimers, homologues, glycosylation pattern variants, variants, splice variants, and mutants, thereof, regardless of the biological activity of same, and further regardless of the method of synthesis or manufacture thereof including, but not limited to, recombinant (whether produced from cDNA, genomic DNA, synthetic DNA or other form of nucleic acid), in vitro, in vivo, by microinjection of nucleic acid molecules, synthetic, transgenic, and gene activated methods.

Accordingly, examples of GH include a growth hormone protein and species and sequence variants thereof, but are not limited to, the 191 single-chain amino acid sequence of human GH. The GH can be the native, full-length protein or can be a truncated fragment or a sequence variant that retains at least a portion of the biological activity of the native protein. There are two known types of human GH derived from the pituitary gland: one having a molecular weight of about 22,000 daltons (22 kD HGH) and the other having a molecular weight of about 20,000 daltons (20 kD HGH). The 20 kD HGH has an amino acid sequence that corresponds to that of 22 kD HGH consisting of 191 amino acids except that 15 amino acid residues from the 32^{nd} to the 46^{th} of 22 kD HGH are missing. Some reports have shown that the 20 kD HGH has been found to exhibit lower risks and higher activity than 22 kD HGH. The disclosure contemplates use of the 22 kD, the 20 kD HGH, as well as species and sequence variants and truncated fragments thereof. The cloned gene for HGH has been expressed in a secreted form in Escherichia coli (U.S. Pat. No. 4,898,830; Chang, C. N., et al., Gene 55:189 [1987]) and its DNA and amino acid sequence has been reported (Goeddel, et al. Nature, 281:544 [1979); Gray, et al., Gene 39: 247[1985]).

Examples of the GH variants suitable here include those having sequences with homology to GH sequences, sequence fragments that are natural, such as from humans, non-human primates, mammals (including domestic animals), and non-natural sequence variants which retain at least a portion of the biologic activity or biological function of GH and/or that are useful for preventing, treating, mediating, or ameliorating a GH-related disease, deficiency, disorder or condition. Non-mammalian GH sequences are well-described in the literature. For example, a sequence alignment of fish GHs can be found in Genetics and Molecular Biology 2003 26 p. 295-300. An analysis of the evolution of avian GH sequences is presented in Journal of Evolutionary Biology 2006 19 p. 844-854. Some exemplary sequences are also described in U.S. Pat. Nos. 8,143,216, 7,662,393, and US 20180161443 which are incorporated by reference. In addition, native sequences homologous to human GH may be found by standard homology searching techniques, such as NCBI BLAST.

In one embodiment, the GH comprises a GH polypeptide with a sequence corresponding to a protein found in nature. In another embodiment, the GH is a sequence variant, fragment, homolog, or a mimetics of a natural sequence that retains at least a portion of the biological activity of the corresponding native GH. Any of these GH sequences or homologous derivatives (e.g., constructed by shuffling individual mutations between species or families) that retain at least a portion of the biological activity of the native GH may be useful for the therapy described herein. Such a GH can exhibit at least about 60%, 70%, 75%, or 80% sequence identity, or preferably 81%, 82%, 83%, 84%, 85%, 86%, 87%, 88%, 89%, 90%, 91%, 92%, 93%, 94%, 95%, 96%, 97%, 98%, 99%, or 100% sequence identity to a GH sequence (e.g., SEQ ID NO: 1).

In general, a GH variant or analogue or derivative exhibits a binding specificity to a given target or another desired biological characteristic when used in vivo or when utilized in an in vitro assay. For example, it may exhibit the ability to bind to a transmembrane receptor for growth hormone. In one embodiment, the binding the growth hormone receptor leads to receptor dimerization and lead to at least a portion of the activation of intercellular signal transduction pathway compared to native growth hormone.

The term “GH polypeptide” encompasses GH polypeptides comprising one or more amino acid substitutions (e.g., particularly conservative modifications or conservative substitutions), additions or deletions. Exemplary substitutions include, e.g., substitution of the lysine at position 41 or the phenylalanine at position 176 of native HGH. In some cases, the substitution may be an isoleucine or arginine residue if the substitution is at position 41 or is a tyrosine residue if the position is 176. Position F10 can be substituted with, e.g., A, H or I. Position M14 may be substituted with, e.g., W, Q or G. Other exemplary substitutions include any substitutions or combinations thereof, including but not limited to:
R167N, D171S, E174S, F176Y, 1179T;
R167E, D171S, E174S, F176Y;
F10A, M14W, H18D, H21N;
F10A, M14W, H18D, H21N, R167N, D171S, E174S, F176Y, I179T;
F10A, M14W, H18D, H21N, R167N, D171A, E174S, F176Y, I179T;
F10H, M14G, H18N, H21N;
F10A, M14W, H18D, H21N, R167N, D171A, T175T, 1179T; or
F10I, M14Q, H18E, R167N, D171S, I179T.
   See, e.g., U.S. Pat. No. 6,143,523, which is incorporated by reference herein.

Exemplary substitutions (e.g., particularly conservative modifications or conservative substitutions) in a wide variety of amino acid positions in naturally-occurring HGH have been described, including substitutions that increase agonist activity, increase protease resistance, convert the polypeptide into an antagonist, etc. and are encompassed by the term “HGH polypeptide.”

Agonist G H, e.g., HGH sequences include, e.g., the naturally-occurring HGH sequence comprising the following modifications H18D, H21N, R167N, D171S, E174S, I179T. See, e.g., U.S. Pat. No. 5,849,535, which is incorporated by reference herein. Additional agonist HGH sequences include H18D, Q22A, F25A, D26A, Q29A, E65A, K168A, E174S; H18A, Q22A, F25A, D26A, Q29A, E65A, K168A, E174S; or H18D, Q22A, F25A, D26A, Q29A, E65A, K168A, E174A. See, e.g. U.S. Pat. No. 6,022,711, which is incorporated by reference herein. HGH polypeptides comprising substitutions at H18A, Q22A, F25A, D26A, Q29A, E65A, K168A, E174A enhance affinity for the HGH receptor at site I. See, e.g. U.S. Pat. No. 5,854,026, which is incorporated by reference herein. HGH sequences with increased resistance to proteases include, but are not limited to, HGH polypeptides comprising one or more amino acid substitutions within the C-D loop. In some embodiments, substitutions include, but are not limited to, R134D, T135P, K140A, and any combination thereof. See, e.g., Alam et al. (1998) J. Biotechnol. 65:183-190.

The term “GH polypeptide” also includes the pharmaceutically acceptable salts and prodrugs, and prodrugs of the salts, polymorphs, hydrates, solvates, biologically-active fragments, biologically active variants and stereoisomers of the naturally-occurring GH as well as agonist and mimetic variants of the naturally-occurring HGH and polypeptide fusions thereof. Fusions comprising additional amino acids at the amino terminus, carboxyl terminus, or both, are encompassed by the term “GH polypeptide.” Exemplary fusions include, but are not limited to, e.g., methionyl growth hormone in which a methionine is linked to the N-terminus of GH resulting from the recombinant expression of the mature form of HGH lacking the secretion signal peptide or portion thereof, fusions for the purpose of purification (including, but not limited to, to poly-histidine or affinity epitopes), fusions with serum albumin binding peptides and fusions with serum proteins such as serum albumin.

The term “GH polypeptide” includes polypeptides conjugated to a polymer such as PEG and may be comprised of one or more additional derivitizations of cysteine, lysine, or other residues. In addition, the GH polypeptide may comprise a linker or polymer, wherein the amino acid to which the linker or polymer is conjugated may be a non-natural amino acid, or may be conjugated to a naturally encoded amino acid utilizing techniques known in the art such as coupling to lysine or cysteine. Polymer conjugation of GH polypeptides has been reported.

See, e.g. U.S. Pat. Nos. 5,849,535, 6,136,563 and 6,608,183, The term “GH polypeptide” also includes glycosylated GH, as well as but not limited to, polypeptides glycosylated at any amino acid position, N-linked or O-linked glycosylated forms of the polypeptide. Variants containing single nucleotide changes are also considered as biologically active variants of GH polypeptide. In addition, splice variants are also included. The term “GH polypeptide” also includes GH, e.g., GH polypeptide heterodimers, homodimers, heteromultimers, or homomultimers of any one or more GH, e.g., GH polypeptides or any other polypeptide, protein, carbohydrate, polymer, small molecule, linker, ligand, or other biologically active molecule of any type, linked by chemical means or expressed as a fusion protein, as well as polypeptide analogues containing, for example, specific deletions or other modifications yet maintain biological activity.

In some embodiments, the GH, e.g., HGH polypeptides, further comprise an addition, substitution or deletion that modulates biological activity of the GH or HGH polypeptide. For example, the additions, substitutions or deletions may modulate one or more properties or activities of GH, e.g., HGH. For example, the additions, substitutions or deletions may modulate affinity for the GH, e.g., HGH polypeptide receptor, modulate (including but not limited to, increases or decreases) receptor dimerization, stabilize receptor dimers, circulating half-life, modulate therapeutic half-life, modulate stability of the polypeptide, modulate cleavage by proteases, modulate dose, modulate release or bio-availability, facilitate purification, or improve or alter a particular route of administration. Similarly, GH, e.g., HGH polypeptides may comprise protease cleavage sequences, reactive groups, antibody-binding domains (including but not limited to, FLAG or poly-His) or other affinity based sequences (including but not limited to, FLAG, poly-His, GST, etc.) or linked molecules (including but not limited to, biotin) that improve detection (including but not limited to, GFP), purification or other traits of the polypeptide.

In some embodiments, one can use any recombinant HGH that is currently sold as a daily injectable product, including HUMATROPE (ELI LILLY & CO.), NUTROPIN (GENENTECH), NORDITROPIN (NOVO-NORDISK), GENOTROPIN (PFIZER), and SAIZEN/SEROSTIM (SERONO). In the US, the following are available, GENOTROPIN, GENOTROPIN MINIQUICK, HUMATROPE, NORDITROPIN FLEXPRO, NUTROPIN AQ NUSPIN 10, NUTROPIN AQ NUSPIN 20, NUTROPIN AQ NUSPIN 5, OMNITROPE, SAIZEN, SAIZENPREP, SEROSTIM, ZOMACTON, ZOMACTON (FOR ZOMA-JET 10) [DSC], and ZORBTIVE. In Canada, the following are available: GENOTROPIN GOQUICK, GENOTROPIN MINIQUICK, HUMATROPE, NORDITROPIN NORDIFLEX PEN, NUTROPIN AQ NUSPIN 10, NUTROPIN AQ NUSPIN 20, NUTROPIN AQ NUSPIN 5, OMNITROPE, SAIZEN, and SEROSTIM.

### Assessments and Evaluations

The method or use disclosed herein can further comprise a step identifying a subject in need of a treatment or evaluating the subject. One or more of these steps can be carried out before or after administering a therapy disclosed herein. Various assessments and tests known in the art can be used for the identifying or evaluating step. Some examples are described below.

### Efficacy Assessments—Neuromuscular Assessments

6 Minute Walk Test: The six minute walk test (6MWT) measure has been extensively studied and utilized in neuromuscular populations. See, e.g., Kierkegaard M, et al. Neuromuscul Disord 2007; 17:943-9; McDonald C M, et al. Muscle Nerve 2010; 42:966-74, and McDonald C M, et al. Muscle Nerve 2010; 41:500-10. It represents patient function and one of the most prevalent issues that impairs FSHD quality-of-life. See, e.g., Heatwole C, et al. Neurology 2012; 78:S15.004.

Quantitative Muscle Testing (QMT): Maximum Voluntary Isometric Contraction Testing of the limb muscles can be performed using the Quantitative Muscle Assessment (QMA) system. This system uses an adjustable cuff to attach the patient's arm or leg to an inelastic strap that is connected to a force transducer with a load of 0.5 to 1,000 Newtons. Measurements resulting from this method of strength testing have been used for several neuromuscular diseases. See, e.g., Kissel J T, et al. Neurology 2001; 57:1434-40; Personius K E, et al. The FSH DY Group. Phys Ther 1994; 74:253-63; Andres P L, Skerry L M, Munsat T L. Measurement of Strength in Neuromuscular Diseases. In: Munsat T L, ed. Quantification of neurologic deficit. Stoneham: Butterworths Publishers, 1989:87-100; and Muscle Study Group. A randomized, pilot trial of etanercept in dermatomyositis. Ann Neurol 2011; 70:427-36.

Six selected muscle groups can be tested bilaterally (biceps, triceps, quadriceps, hamstrings, handgrip, and shoulder external rotators). These particular muscles are chosen because they show excellent test-retest reliability in neuromuscular patients and normal volunteers and they reflect affected muscle groups in FSHD amenable to therapeutic intervention (Kissel J T, et al. Neurology 2001; 57:1434-40). A composite QMT score can be generated by expressing each muscle strength score as a percent of predicted normal given the subject's age, gender, and height and averaging across muscles. See, e.g., Kissel J T, et al. Neurology 2001; 57:1434-40 and Tawil R, et al. Neurology 1997; 48:46-9. Additional analysis of QMT strength can include absolute strength analysis, and analysis of upper and lower limb strength individually.

Manual Muscle Testing (MMT): Manual muscle testing can be performed on 30 muscle groups (bilateral shoulder abductors, shoulder external rotators, elbow flexors, wrist flexors, wrist extensors, hip flexors, hip abductors, knee extensors, hip extensors, knee flexors, hip adductors, elbow extensors, ankle dorsiflexors, and plantar flexors, plus neck extensor and neck flexors). Measurements resulting from this method of strength testing have been utilized in prior clinical trials of musculoskeletal disease. See, e.g., Personius K E, et al. The FSH DY Group. Phys Ther 1994; 74:253-63; Muscle Study Group. A randomized, pilot trial of etanercept in dermatomyositis. Ann Neurol 2011; 70:427-36; Florence J M, et al. Phys Ther 1992; 72:115-22; and Griggs R C, et al. Arch Neurol 1991; 48:383-8.

The FSHD Clinical Outcome Measure (FSHD-COM): The FSHD-COM is an evaluator-administered test that measures multiple aspects of physical function known to be impaired in FSHD. Tests included in the FSHD-COM evaluate leg function, arm/shoulder function, trunk function, hand function, and balance. Leg function is evaluated using sit to stand times (Wagner K R, et al. Ann Neurol 2008; 63:561-71), the six minute walk test, self-selected gait speed (Bohannon R W. J Geriatr Phys Ther 2006; 29:64-8 and Iosa M, et al. Clin Biomech (Bristol, Avon) 2007; 22:1074-82), time to go 30 feet, and time to ascend stairs (Personius K, et al. Phys Ther 1994; 74:253-63). Arm and shoulder function are measured using range of motion determinations at the shoulders and elbows (Anonymous A prospective, quantitative study of the natural history of facioscapulohumeral muscular dystrophy (FSHD): implications for therapeutic trials. The FSH-DY Group. Neurology 1997; 48:38-46), and the time it takes a subject to don and doff a coat (Brown M, et al., J Gerontol A Biol Sci Med Sci 2000; 55:M350-5). Trunk function is measured using the time it takes a subject to pick up a penny from the floor (Brown M, et al., J Gerontol A Biol Sci Med Sci 2000; 55:M350-5), sit up with the feet held, and go from a supine to sitting position. Hand function is measured using QMT grip strength determinations. Lastly, balance is approximated using the timed up and go test (Podsiadlo D, et al., J Am Geriatr Soc 1991; 39:142-8). Each of individual tests of the FSHD-COM (and the FSHD-COM total score) can be analyzed.

### Efficacy Assessments—Functional Assessments

Forced Vital Capacity: Respiratory function can be measured using forced vital capacity.

Epworth Sleepiness Scale can be completed to estimate subject daytime sleepiness.

Fatigue Severity Scale can be completed to estimate subject fatigue.

Dual Energy X-Ray Absorptiometry (DEXA). Lean body mass (LBM) can be measured via Dual Energy X-Ray Absorptiometry (DEXA). DEXA provides a practical and effective approach to determine lean muscle mass and has been extensively utilized in prior neuromuscular clinical trials. See e.g., Logigian E L, et al. Neurology 2010; 74:1441-8 and Heatwole C R, et al. Arch Neurol 2011; 68:37-44.

Both total LBM and regional LBM can be measured (Skalsky A J, et al. Neuromuscul Disord 2008; 18:873-80). DEXA measurement has multiple advantages over the more recently described and debated MRI techniques. Specifically, compared to MRI, DEXA has a more extensive history of use, a more standardized analysis, is not as dependent on perfect subject positioning, and is more accessible, more affordable, and can be utilized in patients with metallic foreign bodies, aneurysm clips, pacemakers, claustrophobia, and metallic implants, and has a better correlation with change in six-minute walk distances compared to MRI thigh measurements. See e.g., Skalsky A J, et al. Phys Med Rehabil Clin N Am 2012; 23:67,73, x and Amato A A, et al. Neurology 2014; 83:2239-46. Most importantly, the use of DEXA as a biomarker is supported by a prior study of combination therapy where LBM increases (measured by DEXA) occurred prior to later observable marked improvements in patient strength and function (Schroeder E T, Eur J Appl Physiol 2012; 112:1123-31).

### Efficacy Assessments Self-reported Assessments

The FSHD-Health Index (FSHD-HI): The FSHD-HI is a disease-specific patient reported outcome measure designed to assess patient-relevant effects during therapeutic trials (Heatwole C, et al. Neurology 2012; 78:S15.004). The instrument measures a patient's assessment of their: (1) Mobility; (2) Hand and arm function; (3) Emotional issues; (4) Cognitive impairment; (5) Decreased satisfaction in social situations; (6) Decreased performance in social situations; (7) Specific activity impairment; (8) Fatigue; (9) Pain; (10) Eating problems; (11) Communication difficulty; (12) Problems with shoulders and arms; (13) Weakness of the back, chest, or abdomen; and, (14) Body image and has been created to satisfy FDA recommendations for use in supporting drug labelling applications. The FSHD-HI is currently being validated in NIH sponsored longitudinal study of 40 FSHD patients at the University of Rochester.

PROMIS-57: The PROMIS-57 generates scores for depression, anxiety, fatigue, pain interference, pain intensity, physical function, sleep disturbance, and satisfaction with participation in social roles (Dunn-Lewis C, et al. Nutr J 2011; 10:90).

Individualized Neuromuscular Quality of Life Questionnaire (INQoL): The INQoL is a muscle disease focused quality-of-life instrument (Vincent K A, et al. Neurology 2007; 68:1051-7).

Beck's Depression Inventory (BDI): The BDI is a widely used instrument for monitoring change in depressive symptoms (BECK A T, et al. Arch Gen Psychiatry 1961; 4:561-71).

### Kit

Also encompassed by the disclosure are kits (e.g., pharmaceutical packs). In certain embodiments, the kit comprises one or more pharmaceutical compositions described herein, and instructions for using the pharmaceutical composition(s).

In some embodiments, the kit comprises two containers containing a GH/GH derivative and a testosterone/testosterone derivative, respectively. In some embodiments, the kit comprises one or more pharmaceutical compositions or compounds described herein and a first container (e.g., a vial, ampule, bottle, syringe, and/or dispenser package, or other suitable container), wherein the first container includes a first pharmaceutical composition (e.g., a GH/GH derivative or a testosterone/testosterone). In certain embodiments, the kit may optionally further include a second container (e.g., a vial, ampule, bottle, syringe, and/or dispenser package, or other suitable container). In some embodiments, the second container comprises a pharmaceutical excipient for dilution or suspension of a pharmaceutical composition or compound described herein. In certain embodiments, the pharmaceutical composition or compound described herein provided in the first container and the second container are combined to form one-unit dosage form. In some embodiments, the kit further comprises instructions for use, e.g., instructions for combining the container components, and/or instructions for administering the container components to a subject.

In certain embodiments, the instructions are for administering the pharmaceutical composition to a subject in need thereof. In certain embodiments, the instructions comprise information required by a regulatory agency, such as the U.S. Food and Drug Administration (FDA) or the European Agency for the Evaluation of Medicinal Products (EMA). In certain embodiments, the instructions comprise prescribing information.

### Definitions

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

A “recombinant” peptide, polypeptide, or protein refers to a peptide, polypeptide, or protein produced by recombinant DNA techniques; i.e., produced from cells transformed by an exogenous DNA construct encoding the desired peptide. A “synthetic” peptide, polypeptide, or protein refers to a peptide, polypeptide, or protein prepared by chemical synthesis. The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Within the scope of this invention are fusion proteins containing one or more of the afore-mentioned sequences and a heterologous sequence. A heterologous polypeptide, nucleic acid, or gene is one that originates from a foreign species, or, if from the same species, is substantially modified from its original form. Two fused domains or sequences are heterologous to each other if they are not adjacent to each other in a naturally occurring protein or nucleic acid.

A conservative modification or functional equivalent of a peptide, polypeptide, or protein disclosed in this invention refers to a polypeptide derivative of the peptide, polypeptide, or protein, e.g., a protein having one or more point mutations, insertions, deletions, truncations, a fusion protein, or a combination thereof. It retains substantially the activity to of the parent peptide, polypeptide, or protein (such as those disclosed in this invention). In general, a conservative modification or functional equivalent is at least 60% (e.g., any number between 60% and 100%, inclusive, e.g., 60%, 70%, 75%, 80%, 85%, 90%, 95%, 96%, 97%, 98%, and 99%) identical to a parent (e.g., SEQ ID NO: 1). Accordingly, within scope of this invention are hinge regions having one or more point mutations, insertions, deletions, truncations, a fusion protein, or a combination thereof.

As used herein, the percent homology between two amino acid sequences is equivalent to the percent identity between the two sequences. The percent identity between the two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below. The percent identity between two amino acid sequences can be determined using the algorithm of E. Meyers and W. Miller (Comput. Appl. Biosci., 4:11-17 (1988)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. 48:444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

As used herein, the term “conservative sequence modifications” refers to amino acid modifications that do not significantly affect or alter the GH receptor binding characteristics of the GH containing the amino acid sequence. Conservative amino acid substitutions are ones in which the amino acid residue is replaced with an amino acid residue having a similar side chain. Families of amino acid residues having similar side chains have been defined in the art.

Amino acid substitutions can be made, in some cases, by selecting substitutions that do not differ significantly in their effect on maintaining (a) the structure of the peptide backbone in the area of the substitution, (b) the charge or hydrophobicity of the molecule at the target sit; or (c) the bulk of the side chain. For example, naturally occurring residues can be divided into groups based on side-chain properties; (1) hydrophobic amino acids (norleucine, methionine, alanine, valine, leucine, and isoleucine); (2) neutral hydrophilic amino acids (cysteine, serine, threonine, asparagine, and glutamine); (3) acidic amino acids (aspartic acid and glutamic acid); (4) basic amino acids (histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Examples of substitutions include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenylalanine, glycine for proline, threonine for serine, serine for threonine, tyrosine for tryptophan, phenylalanine for tyrosine, and/or leucine for valine. Exemplary substitutions are shown in the table below. Amino acid substitutions may be introduced into human erythropoietin and the products screened for retention of the biological activity of human erythropoietin.

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

“Prodrug” or “pharmaceutically acceptable prodrug” refers to a compound that is metabolized, for example hydrolyzed or oxidized, in the host after administration to form the compound of the present disclosure (e.g., compounds of Formula (I), (II), or (III)). The present disclosure includes within its scope, prodrugs of the compounds described herein. Such examples include, but are not limited to, choline ester derivatives and the like, N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C1-C8 alkyl, C2-C8 alkenyl, C2-C8 alkynyl, aryl, C7-C12 substituted aryl, and C7-C12 arylalkyl esters of the compounds described herein may be preferred. Examples of prodrugs of testosterone include, but are not limited to, testosterone undecanoate, testosterone cypionate, testosterone enanthate, testosterone propionate, and testosterone buciclate. Conventional procedures for the selection and preparation of suitable prodrugs are described, for example, in “Design of Prodrugs” Ed. H. Bundgaard, Elsevier, 1985. As used herein, “prodrug” may also refer to a naturally occurring precursor of testosterone, such as androstenedione.

Where the compound disclosed herein is an ester (e.g., testosterone acetate) or prodrug of a compound of Formula(I), (II), or (III) (e.g., testosterone undecanoate), it is understood that the ester or prodrug component (e.g., for testosterone undecanoate, the undecylate ester) may comprise additional instances of hydrogen that are not depicted in the drawing of Formula (I), (II), or (III).

The term “biologically active metabolite” means a pharmacologically active product produced through metabolism in the body of a specified compound (e.g., a compound of Formula (I), (II), or (III) or salt thereof.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄⁻ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “effective amount” as used herein refers to the amount of, e.g. testosterone or GH needed to alleviate at least one or more symptom of the disease or disorder, and relates to a sufficient amount of pharmacological composition to provide the desired effect. The term “therapeutically effective amount” therefore refers to an amount of, e.g., testosterone or GH that is sufficient to provide a beneficial effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of testosterone or GH, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for free testosterone or GH, among others. The dosage can be determined by a physician and adjusted, as necessary, to suit observed effects of the treatment.

The terms “decrease,” “reduced”, “reduction”, and “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. A decrease can be preferably down to a level accepted as within the range of normal for an individual without a given disorder.

The terms “improve,” “increased”, “increase”, “enhance”, or “activate” are all used herein to mean an increase by a statically significant amount. In some embodiments, the terms “improve,” “increased,” “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” or “individual” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, sheep, goats, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a human or a non-human mammal. The mammal can be a human, non-human primate, mouse, rat, dog, cat, horse, or cow, but is not limited to these examples. Mammals other than humans can be advantageously used as subjects that represent animal models of disorders. The terms, “individual,” “patient” and “subject” are used interchangeably herein. A subject can be male or female.

A subject can be one who has been previously diagnosed with or identified as suffering from or having a condition or disorder in need of treatment (e.g., FSHD) or one or more complications related to such a condition or disorder, and optionally, have already undergone treatment for such a condition or disorder or the one or more complications related to the condition or disorder. Alternatively, a subject can also be one who has not been previously diagnosed as having a condition or disorder or one or more complications related to the condition or disorder. For example, a subject can be one who exhibits one or more risk factors for the condition or disorder or one or more complications related to the condition or disorder or a subject who does not exhibit risk factors.

A “subject in need” of treatment for a particular condition or disorder can be a subject having that condition or disorder, diagnosed as having that condition or disorder, or at risk of developing that condition or disorder.

As used herein, the term “administering,” refers to the placement of an agent (e.g., GH or testosterone or a derivative thereof) as disclosed herein into a subject by a method or route which results in at least partial delivery of the agent at a desired site. Pharmaceutical compositions comprising the agents disclosed herein can be administered by any appropriate route which results in an effective treatment in the subject. The terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. FSHD. The term “treating” includes reducing or alleviating at least one adverse effect or symptom of a condition, disease or disorder associated with a disorder. Treatment is generally “effective” if one or more symptoms or clinical markers are reduced. Alternatively, treatment is “effective” if the progression of a disease is reduced. That is, “treatment” includes not just the improvement of symptoms or markers, but also a slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

A “therapeutically effective amount” is an amount sufficient to remedy a disease state or symptoms, particularly a state or symptoms associated with the disease state, or otherwise prevent, hinder, retard or reverse the progression of the disease state or any other undesirable symptom associated with the disease in any way whatsoever.

A “prophylactically effective amount” is an amount of a pharmaceutical composition that, when administered to a subject, will have the intended prophylactic effect, e.g., preventing or delaying the onset (or reoccurrence) of the disease state, or reducing the likelihood of the onset (or reoccurrence) of the disease state or associated symptoms. The full therapeutic or prophylactic effect does not necessarily occur by administration of one dose, and may occur only after administration of a series of doses. Thus, a therapeutically or prophylactically effective amount may be administered in one or more administrations.

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable carrier or excipient” refers to a carrier medium or an excipient which does not interfere with the effectiveness of the biological activity of the active ingredient(s) of the composition and which is not excessively toxic to the host at the concentrations at which it is administered. In the context of the present invention, a pharmaceutically acceptable carrier or excipient is preferably suitable for topical formulation. The term includes, but is not limited to, a solvent, a stabilizer, a solubilizer, a tonicity enhancing agent, a structure-forming agent, a suspending agent, a dispersing agent, a chelating agent, an emulsifying agent, an anti-foaming agent, an ointment base, an emollient, a skin protecting agent, a gel-forming agent, a thickening agent, a pH adjusting agent, a preservative, a penetration enhancer, a complexing agent, a lubricant, a demulcent, a viscosity enhancer, a bioadhesive polymer, or a combination thereof. The use of such agents for the formulation of pharmaceutically active substances is well known in the art (see, for example, “Remington's Pharmaceutical Sciences”, E. W. Martin, 18th Ed., 1990, Mack Publishing Co.: Easton, PA, which is incorporated herein by reference in its entirety).

As used herein, the term “a,” “an,” “the” and similar terms used in the context of the present invention (especially in the context of the claims) are to be construed to cover both the singular and plural unless otherwise indicated herein or clearly contradicted by the context.

Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided herein is intended merely to better illuminate the invention and does not pose a limitation on the scope of the invention otherwise claimed. No language in the specification should be construed as indicating any non-claimed element essential to the practice of the invention.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The term “about” refers to within 10%, preferably within 5%, and more preferably within 1% of a given value or range. Alternatively, the term “about” refers to within an acceptable standard error of the mean, when considered by one of ordinary skill in the art.

As used herein the term “comprising” or “comprises” is used in reference to compositions, methods, and respective component(s) thereof, that are essential to the method or composition, yet open to the inclusion of unspecified elements, whether essential or not. The term “consisting of” refers to compositions, methods, and respective components thereof as described herein, which are exclusive of any element not recited in that description of the embodiment. As used herein the term “consisting essentially of” refers to those elements required for a given embodiment. The term permits the presence of elements that do not materially affect the basic and novel or functional characteristic(s) of that embodiment.

### EXAMPLES

The examples describe a proof-of-concept, single-center, open-label study of daily human growth hormone (Genotropin®, 5.0 μg/kg via subcutaneous injection) and testosterone (testosterone enanthate, 140 mg via intramuscular injection every two weeks) for 4 months in men with FSHD. A total of 20 subjects were enrolled in this study at the University of Rochester Medical Center in Rochester, NY.

### Example 1

This example describes study objectives and measures, investigational plan, and selection and enrollment of subjects.

A primary objective of the study was to examine the safety and tolerability of rHGH and testosterone in adult male patients with FSHD. Secondary objectives included (i) to examine the pharmacokinetic effects of rHGH and testosterone on serum levels of free and total testosterone, IGF-1, thyroid function, luteinizing hormone and follicle stimulating hormone and (ii) to examine the effect of rHGH and testosterone on total and regional lean body mass. Exploratory objectives included to examine the effects of rHGH and testosterone on multiple measures of clinical function including ambulation, strength, pulmonary function, and patient reported disease-burden.

Safety outcome measures included (1) Changes from baseline in laboratory test results, vital signs, and EKG results, (2) Occurrence of serious adverse events, and (3) Occurrence of non-serious adverse events. Exploratory efficacy outcome measures included the following:
1. Composite Quantitative Muscle Testing (QMT) score
2. Composite Manual Muscle Testing (MMT) score
3. FSHD Clinical Outcome Measure (FSHD-COM), total and individual test scores
4. FSHD-Health Index (FSHD-HI), disease-specific patient-reported outcome measure
5. Forced Vital Capacity (FVC)
6. Epworth Sleepiness Scale
7. Fatigue Severity Scale
8. Dual Energy X-Ray Absorptiometry (DEXA) Lean Body Mass (total and regional)
9. PROMIS-57
10. Individualized Neuromuscular Quality of Life Questionnaire (INQoL)
11. Beck Depression Inventory (BDI)
12. Six minute walk distance

Participants received the following intervention: (1) 5.0 μg/kg of rHGH per day (after dinner) by subcutaneous injection; and (2) 140 mg of testosterone enanthate IM given every two weeks. The dosage of the rHGH was calculated using the patient's pre-entry weight. All participants were taught to self-administer the study medications.

The table below provides information on the precision with which the incidence of a particular adverse event can be estimated as well as the probability of observing at least one such event during the trial given different values for the true incidence of the adverse event in the target FSHD population receiving combination therapy. A sample size of 20 participants can yield a 95% upper confidence bound for the incidence that is within approximately 20% of the observed incidence. It can also make it very likely that an adverse event with a true incidence >15% can be observed in at least one subject during the trial.

TABLE 1
Estimation of adverse event incidence with n = 20 participants

| | | | Probability of |
|---|---|---|---|
| | 95% Upper | True Adverse | Observing at |
| Observed Adverse | Confidence | Event | Least 1 |
| Event Incidence | Bound | Incidence | Adverse Event |
| 5% | 21.6% | 5% | 26.1% |
| 10% | 28.3% | 10% | 60.8% |
| 15% | 34.4% | 15% | 82.4% |
| 20% | 40.1% | 20% | 93.1% |
| 25% | 45.6% | 25% | 98.0% |
| 30% | 50.8% | 30% | 99.2% |

With respect to the exploratory measures of efficacy the change from baseline to Week 24 in the total distance walked in 6 minutes (6MWT) was considered. An estimated standard deviation of 42.3 meters was derived using data on 6-month change in 6MWT from 9 participants in an ongoing longitudinal study of FSHD progression. A similar estimate of 38.5 meters was derived using data on 12-month change in 6MWT from 71 participants in a longitudinal study of progression in participants with DM1. Assuming a standard deviation of 40 meters for the primary outcome variable, a sample size of 18 participants can provide 85% power to detect a mean change of 30 meters, using a t-test and a 5% significance level (two-tailed). The mean 6MWT distance at baseline in the FSHD cohort was approximately 350 meters. Therefore, the chosen effect size of 30 meters represents, in relative terms, an approximately 8.6% improvement in 6MWT. Recent studies in chronic obstructive pulmonary disease, coronary artery disease, and Duchenne muscular dystrophy support a distance of 25-35 meters as representing a minimal clinically important change. A sample size of 20 participants can allow for an anticipated attrition rate of 10%.

Analysis of Lean Body Mass: Lean body mass obtained using DEXA was an objective primary marker used to determine whether combination treatment shows sufficient promise to consider further studies in FSHD. The analysis of this outcome involved the use of a repeated measures analysis of variance model (i.e., the so-called “mixed model repeated measures”, or MMRM, analysis strategy, with time (treated as a categorical variable) as the factor of interest. The covariance matrix for the within-subject observations was modeled using an unstructured pattern. Ninety-five percent confidence intervals for mean changes from baseline to each visit was computed using this model, with the 24-week time point being of primary interest. A test for significance of the mean change from baseline to 24 weeks was performed with this model using a significance level of 5% (two-tailed). The analyses was performed according to the intention-to-treat principle and included all enrolled participants. The repeated measures analysis of variance model used for these analyses uses a direct-likelihood approach to estimate the parameters of interest using all available data from all participants. A key assumption underlying this analysis is that the missing data are “missing at random” (MAR), i.e., the probability that the responses are missing for a subject depends only on the set of observed data for that subject and not on the specific missing values that were not obtained. For lean body mass, an estimated standard deviation of 1.6 kg was derived using data on 24-week change from participants in our prior longitudinal study of FSHD progression (PI: Heatwole, U01AR065119). Assuming a standard deviation of 1.6 kg meters for the this outcome variable, a sample size of 18 participants provided a 85% power to detect a mean change of 1.2 kg using a t-test and a 5% significance level (two-tailed).

Experienced investigators were responsible for recruitment and follow-up of 20 subjects for this clinical trial. Participants were moderately affected, ambulatory, FSHD men ages 18 to 65. All patients were symptomatic and had genetically confirmed FSHD or clinical symptoms suggestive of FSHD with a first degree relative with genetically confirmed FSHD. Complete inclusion and exclusion eligibility criteria are listed below. To be eligible for enrollment into this study, subjects must meet all of the following eligibility criteria.

### Inclusion Criteria

Males ages 18 to 65 years
Moderately affected, symptomatic, ambulatory, FSHD (genetically confirmed or clinical symptoms suggestive of FSHD with a first degree relative with genetically confirmed FSHD)
Hematocrit of <50%
Prostate-specific antigen 4.0 ng/ml (or <3.0 ng/ml if the participant is at elevated risk of prostate cancer
Fasting blood glucose <126 mg/dl
Able to walk continuously for six minutes (cane, walker, orthoses allowed) for a distance greater than 75 and less than 700 meters.

### Exclusion Criteria

Diabetes, Obesity (BMI>35 kg/m2), or cardiovascular disease (Heart failure, coronary artery disease, uncontrolled hypertension, untreated hypercholesterolemia)
Untreated thyroid disease
Deep vein thrombosis
Untreated severe sleep apnea
Past pituitary disease
Liver disease
Psychiatric disease
Renal disease
Severe lower urinary tract symptoms (International Prostate Symptom Score (IPSS)>19)
Cancer (other than basal cell skin cancer)
An active desire to conceive, heavy alcohol use (greater than 50 g/day)
Current testosterone or HGH use
Current use of medications that interfere with the growth hormone or gonadal endocrine axis.

Volunteers from diverse ethnic and racial backgrounds were recruited using local and nationwide recruitment tools. These individuals were evaluated at the University of Rochester, which has a history of successfully recruiting patients for FSHD clinical trials.

### Example 2

This example describes drug administration and assignment.

GENOTROPIN® was supplied by PFIZER INC. The generic form of testosterone enanthate were purchased by the investigational pharmacy from a local vendor. Both drugs were received and dispensed by the Investigational Drug Services unit at the University of Rochester.

The participants were given study medication during their baseline visit and subsequently were given 5 weeks of GENOTROPIN® to take home with them in a properly chilled container (to last to their 4 week visit allowing for a one week delay). They were also given their 2 and 4 week testosterone dosages. At the 4 and 8 week study visits, participants were again given enough GENOTROPIN® to last 5 additional weeks from their inpatient visit and testosterone doses were given 2 and 4 weeks later. Prior to week 12 of the study, participants were mailed the appropriate amount of testosterone (given at weeks 12 and 14) and exact amount of GENOTROPIN® to complete the study. Treatment packs were numbered for tracking. Compliance were tracked using a compliance questionnaire and empty packs and vials were collected at each study visit. Subjects self-administered each study drug.

Testosterone enanthate: Subjects received vials of properly dosed testosterone enanthate to be administered as one intramuscular injection into the gluteal muscle. When properly given, intramuscular injections of testosterone enanthate were well tolerated. Care were taken to slowly inject the preparation deeply into the gluteal muscle, following the usual precautions for intramuscular administration, such as the avoidance of intravascular injection.

GENOTROPIN®: Subjects were given GENOTROPIN PEN® 5 delivery devices with 5 mg cartridges. This device had a dial to select the proper dose of study drug and a dose display directly on the Pen. Participants were instructed on how to use the dial to administer the correct medication dose. Participants received GENOTROPIN® using these Pens via subcutaneous injection, as calculated based on body weight at the Screening/Baseline Visit. Each subject pinched a fold of skin at the injection site, inserted the pen at a 90 degree angle, pushed the black/white injection knob until it clicks, waited 5 seconds, then withdraw Pen. Participants were given a document detailing the use of the GENOTROPIN PEN® 5 and taught how to use the Pen during their inpatient visit.

### Example 3

This example describes clinical and laboratory evaluations. An overview of the schedule of activities and evaluation is provided in Table 2.

TABLE 2
Schedule of Study Activities and Testing

| | Screening | Baseline | 4 w | 8 w | 12 w | 16 w | 20 w | 24 w | 36 w |
|---|---|---|---|---|---|---|---|---|---|
| Activity | | | | | | | | | |
| On Combination Therapy | | | | | | | | | |
| Washout Period | | | | | | | | | |
| Informed consent | X | | | | | | | | |
| Documentation of | X | | | | | | | | |
| Inclusion/Exclusion | | | | | | | | | |
| criteria | | | | | | | | | |
| Clinic Visits | X | X | | X | | X | | X | X |
| Laboratory Testing * | X | | | X | | X | | X | X |
| Study Medication | | X | X | X | X | X | X | | |
| Dispensed or Mailed | | | | | | | | | |
| Dual Energy X-Ray | | X | | | | X | | X | X |
| Absorptiometry (DEXA) | | | | | | | | | |
| Six Minute Walk Test | X | | | | | | | | |
| FSHD-COM | | X | | X | | X | | X | X |
| Strength and Functional | | X | | X | | X | | X | X |
| Testing | | | | | | | | | |
| Exercise Log Review | | | | X | | X | | X | X |
| Patient-Reported | X | X | | X | | X | | X | X |
| Outcome Measures | | | | | | | | | |
| Safety Monitoring | | | | | | | | | |
| Vitals ** | X | X | | X | | X | | X | X |
| Physical Exam | X | | | X | | X | | X | X |
| Fundoscopic Exam | | X | | X | | X | | X | X |
| Digital Rectal Exam | X | | | X | | X | | X | X |
| International Prostate | X | | | X | | X | | X | X |
| Symptom Score | | | | | | | | | |
| Quantitative Insulin | | X | | X | | X | | X | X |
| Sensitivity Check Index | | | | | | | | | |
| Side effect questionnaire | | X | | X | | X | | X | X |
| EKG | X | | | X | | X | | X | X |
| Weekly Phone Calls | | | | | | | | | |

* Free and total testosterone levels, free and total IGF-1, PSA, fasting glucose, chem 14, CK, lipid profile, luteinizing hormone, insulin, HGA1c, FSH, CBC, CRP, TSH, free T4, urinalysis
** Height, weight, blood pressure, body mass index, pulse, respiratory rate, temperature

Following informed consent, each participant had a screening visit. Participants who met all eligibility requirements were enrolled and received serial inpatient assessments at baseline, 8 weeks, 16 weeks, 24 weeks and 36 weeks. Throughout the study, participants received weekly phone calls from a clinical coordinator to address any study-related difficulties. All subjects were followed for the 36 weeks, if willing, regardless of protocol violations or withdrawal of study drug.

### Screening and Baseline Evaluations

The Screening and Baseline Evaluations took place at the same visit and required an overnight stay at the Clinical Research Center at the University of Rochester. Screening evaluations took place after the subject had signed the informed consent document. Baseline evaluations took place after the inclusion/exclusion criteria had been fulfilled (e.g. fasting serum glucose, liver function tests, hematocrit, serum prostate specific antigen, and 6 minute walk test).

During this visit, the subject was thoroughly informed about all aspects of the study, including all scheduled visits and activities, and was requested to sign and date the informed consent prior to performing any study-related procedures.

Subjects were assessed for study eligibility by the Investigator or Coordinator. All the inclusion criteria must be met and none of the exclusion criteria may apply. All the results from the screening procedures must be available before determining a subject's eligibility for the study.

The following procedures were performed at the screening visit:
Obtain written informed consent (if not already obtained)
Subject Identification Number assigned
Inclusion/exclusion criteria review
Medical history
Demographics
Review concomitant medication usage
Obtain vital sign measurements (at Obtain vital signs (Height, weight, blood pressure, body mass index, pulse, respiratory rate, waist circumference)
Obtain blood sample for laboratory testing (Total testosterone levels, free testosterone levels, total IGF-1, PSA, fasting glucose, comprehensive metabolic panel, CK, lipid profile, fasting insulin level, TSH, and Free T4 levels, lutenizing hormone, FSH, CBC)
Physical Exam
6 Minute Walk Test
International Prostate Symptom Score
Rectal exam
Perform electrocardiogram (EKG).

The following baseline visit procedures were performed after eligibility criteria had been fulfilled:
Enrollment of subject into study
Conduct final review of eligibility
6 Minute Walk Test
Quantitative Muscle Testing
Manual Muscle Testing
FSHD Clinical Outcome Measure (FSHD-COM)
Forced Vital Capacity
FSHD-Health Index (FSHD-HI)
PROMIS-57
Beck Depression Inventory (BDI)
Fatigue Severity Scale
Dual Energy X-Ray Absorptiometry (DEXA)
Fundoscopic exam
Laboratory studies (if not done within 7 days as part of the screening visit)
Quantitative Insulin Sensitivity Check Index
Side Effect Questionnaire
International Physical Activity Questionnaire
Administration of first dosage of each therapy

After the first dosage of each therapy has been administered, the following activities would occur:
Obtain vital signs (blood pressure, pulse, respiratory rate)
Inquire about any immediate side effects (e.g., nausea, dizziness, GI discomfort)
Complete dose management log to record administration of first doses
Instruct subject about correct administration of study drug
Instruct subjects to bring unused study drug to each study visit, and to immediately report any adverse events to the investigator or coordinator.

### Follow Up Visits

After the subjects had completed the Screening/Baseline Visit, they returned to the Clinical Research Center for an in-person visit at 8 weeks, 16, and 24 weeks of treatment. They were also evaluated at week 36 after 12 weeks of washout. Subjects were contacted weekly to inquire about their general status and resolve any study medication issues. The in-person visits took about 6 hours.

The following activities were completed during the on-site follow up visits. These visits may be conducted within +4 days of the target date.

### Week 8 Visit

An in-person visit at Week 8 involved:
Blood specimen for clinical safety (total testosterone levels, free testosterone levels, total IGF-1, PSA, fasting glucose, comprehensive metabolic panel, CK, lipid profile, HGA1c, fasting insulin level, TSH, and Free T4 levels, LH, FSH, CBC, CRP)
6 Minute Walk Test
Quantitative Muscle Testing
Manual Muscle Testing
FSHD Clinical Outcome Measure (FSHD-COM)
Forced Vital Capacity
FSHD-Health Index (FSHD-HI)
PROMIS-57
Beck Depression Inventory (BDI)
Fatigue Severity Scale
Vital signs (Height, weight, blood pressure, body mass index, pulse, respiratory rate, waist circumference)
Fundoscopic exam
Physical exam
Rectal exam
International Prostate Symptoms Score
Quantitative Insulin Sensitivity Check Index
Side effect questionnaire
International Physical Activity Questionnaire
EKG
Review of concomitant medications and adverse events

Participants with no clinically significant adverse events or lab abnormalities will remain on their daily dosage of study medication. Participants will receive their study medication for the next 4 weeks by postal mail.

### Week 16 Visit

An in-person visit at Week 16 involved:
Blood specimen for clinical safety (Total testosterone levels, free testosterone levels, total IGF-1, PSA, fasting glucose, comprehensive metabolic panel, CK, lipid profile, HGA1c, fasting insulin level, TSH, and Free T4 levels, LH, FSH, CBC, CRP)
6 Minute Walk Test
Quantitative Muscle Testing
Manual Muscle Testing
FSHD Clinical Outcome Measure (FSHD-COM)
Forced Vital Capacity
FSHD-Health Index (FSHD-HI)
PROMIS-57
Beck Depression Inventory (BDI)
Fatigue Severity Scale
Dual Energy X-Ray Absorptiometry (DEXA)
Vital signs (Height, weight, blood pressure, body mass index, pulse, respiratory rate, waist circumference)
Fundoscopic exam
Physical exam
Rectal exam
International Prostate Symptoms Score
Quantitative Insulin Sensitivity Check Index
Side effect questionnaire
International Physical Activity Questionnaire
EKG
Review of concomitant medications and adverse events

Those participants with no clinically significant adverse events or lab abnormalities will remain on their daily dosage of study medication.

### Week 24 Visit

An in-person visit at Week 24 involved:
Blood specimen for clinical safety (Total testosterone levels, free testosterone levels, total IGF-1, PSA, fasting glucose, comprehensive metabolic panel, CK, lipid profile, HGA1c, fasting insulin level, TSH, and Free T4 levels, LH, FSH, CBC, CRP)
6 Minute Walk Test
Quantitative Muscle Testing
Manual Muscle Testing
FSHD Clinical Outcome Measure (FSHD-COM)
Forced Vital Capacity
FSHD-Health Index (FSHD-HI)
PROMIS-57
Beck Depression Inventory (BDI)
Fatigue Severity Scale
Dual Energy X-Ray Absorptiometry (DEXA)
Vital signs (Height, weight, blood pressure, body mass index, pulse, respiratory rate, waist circumference)
Fundoscopic exam
Physical exam
Rectal exam
International Prostate Symptoms Score
Quantitative Insulin Sensitivity Check Index
side effect questionnaire
International Physical Activity Questionnaire
EKG
Review of concomitant medications and adverse events

After this visit, participants will go off of study medication.

Week 36 Visit (12 Weeks after Washout)

An in-person visit at Week 36 involved:
Blood specimen for clinical safety (Total testosterone levels, free testosterone levels, total IGF-1, PSA, fasting glucose, comprehensive metabolic panel, CK, lipid profile, HGA1c, fasting insulin level, TSH, and Free T4 levels, LH, FSH, CBC, CRP)
6 Minute Walk Test
Quantitative Muscle Testing
Manual Muscle Testing
FSHD Clinical Outcome Measure (FSHD-COM)
Forced Vital Capacity
FSHD-Health Index (FSHD-HI)
PROMIS-57
Beck Depression Inventory (BDI)
Fatigue Severity Scale
Dual Energy X-Ray Absorptiometry (DEXA)
Vital signs (Height, weight, blood pressure, body mass index, pulse, respiratory rate, waist circumference)
Fundoscopic exam
Physical exam
Rectal exam
International Prostate Symptoms Score
Quantitative Insulin Sensitivity Check Index
Side effect questionnaire
International Physical Activity Questionnaire
EKG
Review of concomitant medications and adverse events

Participants received a weekly phone call to evaluate for any side effects or interval changes in medical health.

### Example 4

This example describes assessments and other evaluations of the subjects. Efficacy assessments were carried out using the six minute walk test (6MWT) measure, Quantitative Muscle Testing (QMT), Manual Muscle Testing (MMT), and FSHD Clinical Outcome Measure (FSHD-COM). Functional assessments were carried out using Forced Vital Capacity, Epworth Sleepiness Scale, Fatigue Severity Scale, and Dual Energy X-Ray Absorptiometry (DEXA). Self-reported assessments included the FSHD-Health Index (FSHD-HI), PROMIS-57, Individualized Neuromuscular Quality of Life Questionnaire (INQoL), and Beck's Depression Inventory (BDI).

For safety assessments and adverse event assessments, various clinical safety Laboratory tests were carried out. For treatment-specific assessments, the subjects were monitored for the following treatment-related events:
peripheral edema
carpal tunnel syndrome
gynecomastia
insulin resistance
arthralgia
increased erythropoiesis
reduced HDL cholesterol
elevated prostate specific antigen
elevated blood pressure

Physical exams were implemented to evaluate for peripheral edema, carpal tunnel symptoms, and joint pain, and included a fundoscopic examination using a pan-optic ophthalmoscope. Participants were also be monitored with the International Prostate Symptom Scale (Barry M J, et al. The American Urological Association symptom index for benign prostatic hyperplasia. The Measurement Committee of the American Urological Association. J Urol 1992; 148:1549,57; discussion 1564), and the quantitative insulin sensitively check index (Quicki) to evaluate for signs of prostate dysfunction or diabetes. See Blackman M R, et al. JAMA 2002; 288:2282-92, and Sattler F R, et al. J Clin Endocrinol Metab 2009; 94:1991-2001. Patients serially completed a treatment-specific questionnaire at each study visit, designed for early detection of adverse events associated with study drug and were contacted on a weekly basis by study personnel to monitor for any adverse events (Giannoulis M G, et al. J Clin Endocrinol Metab 2006; 91:477-84).

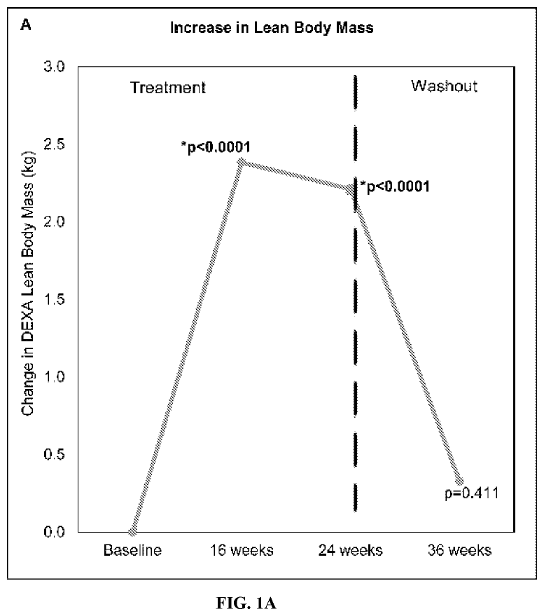

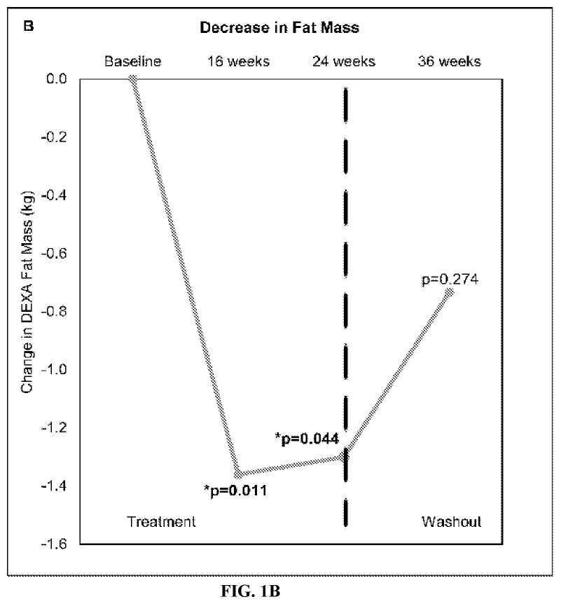

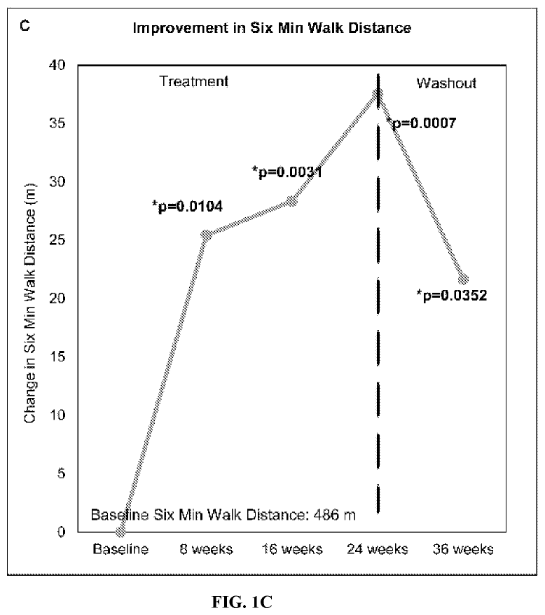

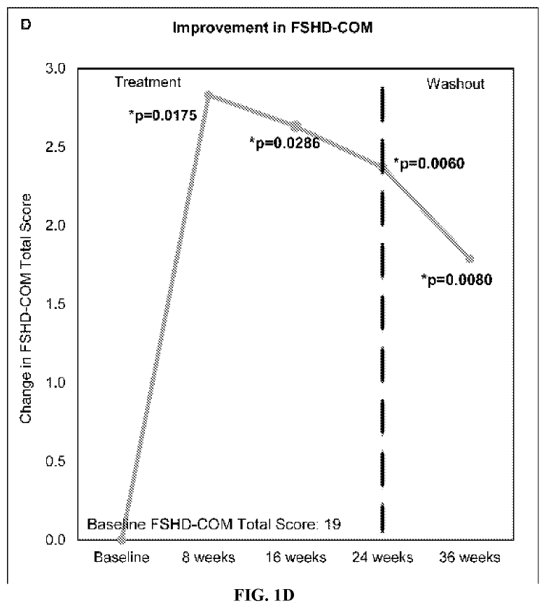

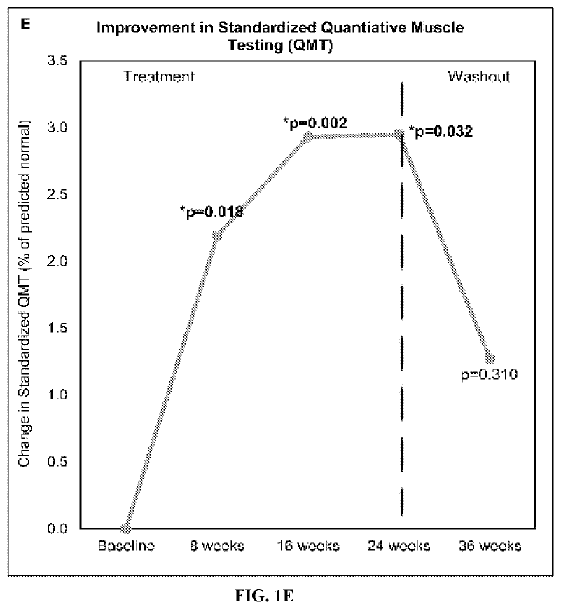

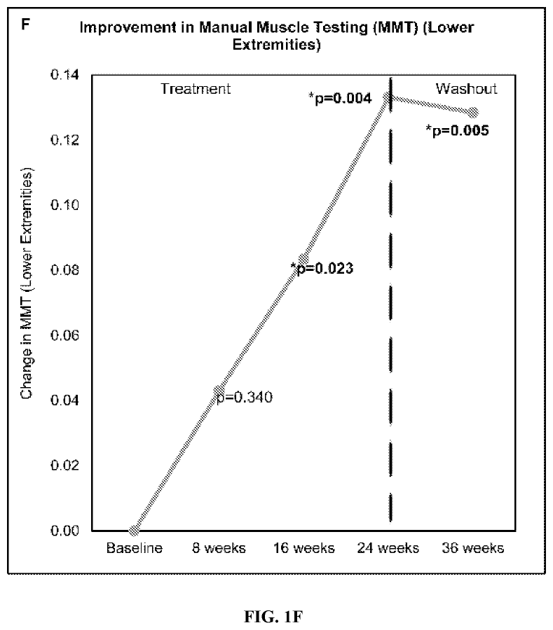

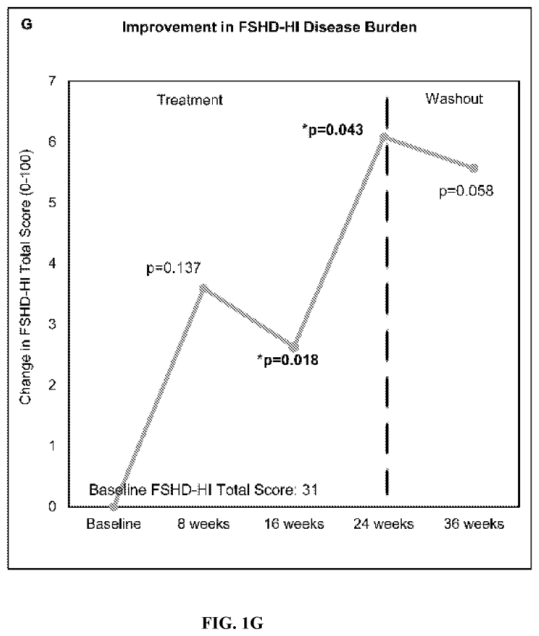

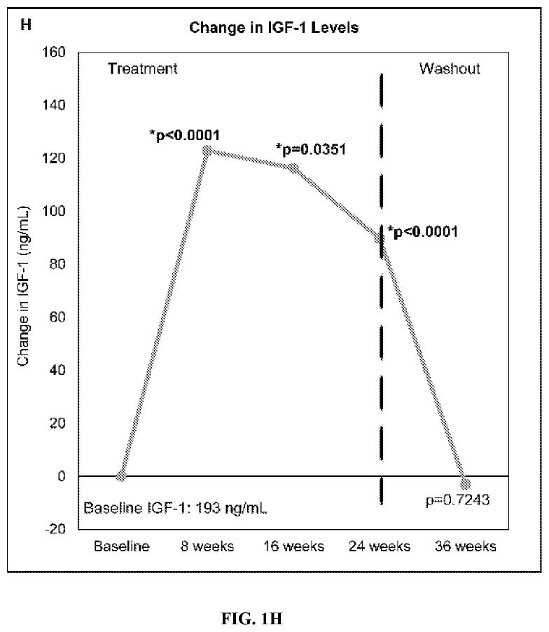

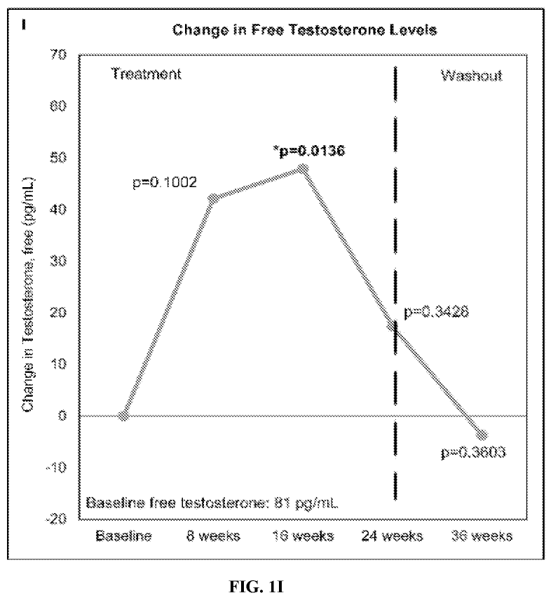

As shown in FIGS. 1A-1I, results of the above assessments indicated various statistical and clinically relevant improvements after 24 weeks of treatments. The average six-minute walk distance increased by 37.6 meters (p=0.001), two-minute walk distance increased by 11.6 meters (p=0.03), total lean body mass (measure by DEXA) increased by 2.2 kg (p<0.0001), total body fat (measured by DEXA) decreased by 1.3 kg (p=0.044), overall strength from 6 bilateral muscle groups (standardized QMT, average % of predicted normal) increased by 5.5% (p=0.032), upper body strength from 4 bilateral muscle groups (standardized QMT, average % of predicted normal) increased by 4.9% (p=0.031), average lower extremity strength from 8 bilateral muscle groups (manual muscle testing) increased by 3.0% (0.13 points; p=0.004), physical function measured by the FSHD-COM improved by 12.4% (2.4 points; p=0.006), and Beck's Depression Inventory score improved by 31.6% (1.7 points; p=0.031). With regard to PROMIS-57 scores, the fatigue score improved by 10.6% (5.7 points; p=0.002) and the pain interference score improved by 11.7% (6.5 points; p=0.005) from baseline. In terms of FSHD-HI scores, the total score improved by 19.5% (6.08 points; p=0.043), the shoulder/arm function score improved by 23.7% (11.4 points; p=0.024), the core strength/function score improved by 30.3% (10.4 points, p=0.018), the fatigue score improved by 29.9% (11.5 points; p=0.017), and the pain score improved by 31.0% (9.5 points; p=0.011) from baseline. These changes, importantly, are correlated with the change in IGF-1 levels from baseline: IGF-1 levels increased by 46.5% (89.79 ng/mL; p<0.0001) from baseline.

TABLE 3
Summary of Effects of Testosterone and rHGH on FSHD Patients at 24 Weeks

| Outcome | Improvement | | p-value |
|---|---|---|---|
| Six-minute walk distance | 37.6 meters | (7.7% improvement) | p = 0.001 |
| Two-minute walk distance | 11.6 meters | (7.1% improvement) | p = 0.03 |
| Total lean body mass (DEXA) | 2.2 kg increase | (4.3% increase) | p < 0.0001 |
| Total fat mass (DEXA) | 1.3 kg reduction | (4.5% reduction) | p = 0.044 |
| Overall muscle strength (standardized | 2.95% improvement | | p = 0.032 |
| QMT) | | | |
| Upper body muscle strength (standardized | 2.78% improvement | | p = 0.031 |
| QMT) | | | |
| Lower body muscle strength (MMT) | 0.13 points | (3.0% improvement) | p = 0.004 |
| Physical function (FSHD-COM) | 2.4 points | (12.4% improvement) | p = 0.006 |
| Beck's Depression Inventory score | 1.7 points | (31.6% improvement) | p = 0.031 |
| PROMIS-57 Fatigue subscale | 5.7 points | (10.6% improvement) | p = 0.002 |
| PROMIS-57 Pain Interference subscale | 6.5 points | (11.7% improvement) | p = 0.005 |
| FSHD-HI Total score | 6.1 points | (19.5% improvement) | p = 0.043 |
| FSHD-HI Shoulder/Arm Function | 11.4 points | (23.7% improvement) | p = 0.024 |
| subscale | | | |
| FSHD-HI Core Strength/Function subscale | 10.4 points | (30.3% improvement) | p = 0.018 |
| FSHD-HI Fatigue subscale | 11.5 points | (29.9% improvement) | p = 0.017 |
| FSHD-HI Pain subscale | 9.5 points | (31.0% improvement) | p = 0.011 |
| IGF-1 level | 89.79 ng/mL | (46.5% increase) | p < 0.0001 |

These unexpected superior results demonstrate that, in a clinical trial, the combination therapy of testosterone and growth hormone has a significant benefit in functional ability, disease burden, muscle mass, fat reduction, ambulation, fatigue, pain, and strength for patients with FSHD. The scope of this benefit has never been demonstrated using any other therapeutic mechanisms in FSHD.

The foregoing examples and description of the preferred embodiments should be taken as illustrating, rather than as limiting the present disclosure as defined by the claims. As will be readily appreciated, numerous variations and combinations of the features set forth above can be utilized without departing from the present disclosure as set forth in the claims. Such variations are not regarded as a departure from the scope of the disclosure, and all such variations are intended to be included within the scope of the following claims. All references cited herein are incorporated by reference in their entireties.

1. A method for (i) improving muscle mass, muscle strength, or muscle function or (ii) treating a disorder associated with muscle wasting or muscle weakness, or (iii) reducing fatigue, pain, or obesity in a subject in need thereof, the method comprising: administering to the subject an effective amount of testosterone or a derivative thereof, and administering to the subject an effective amount of growth hormone or a derivative thereof.

2. (canceled)

3. The method of claim 1, wherein the subject is healthy, has a disorder or condition associated with muscle wasting or muscle weakness, or is at risk of developing the disorder or condition.

4. The method of claim 3, wherein the disorder or condition is selected from the group consisting of Facioscapulohumeral muscular dystrophy (FSHD), Sarcopenia, Duchenne Muscular dystrophy, Limb Girdle Muscular dystrophy, Becker's Muscular dystrophy, Pompe disease, Myotonic dystrophy type-1, myotonic dystrophy type-2, inclusion body myositis, polymyositis, dermatomyositis, ALS, spinal muscular atrophy, Charcot Marie Tooth disease, HIV myopathy, wasting of the elderly, deconditioning, nutritional deficiency, injury, wasting associated with cancer, muscle dysfunction, nerve dysfunction, neuromuscular junction dysfunction, and motor neuron disease.

5. The method of claim 4, wherein the disorder is FSHD.

6. The method of claim 1, wherein the derivative is a testosterone ester.

7. The method claim 6, wherein the derivative is one having structure of Formula (II): 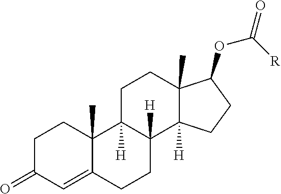
wherein R is alkyl, alkanediyl, alkenyl, alkenediyl, alknyl, aralkyl, aryl, heteroaryl, or acyl.

8. The method of claim 7, wherein the testosterone derivative is selected from the group consisting of testosterone enanthate, testosterone propionate, testosterone cypionate, testosterone undecanoate, testosterone oleate, and testosterone palmitate.

9. The method of claim 8, wherein the derivative is testosterone enanthate.

10. The method of claim 9, wherein the testosterone or derivative thereof is administered at about 0.1 mg to about 30,000 mg.

11. The method of claim 10, wherein the testosterone or derivative thereof is administered at about 70 mg to about 170 mg once every two weeks or at about 110 mg to about 150 mg once every two weeks.

12. (canceled)

13. The method of claim 11, wherein the testosterone or derivative thereof is administered at about 140 mg once every two weeks.

14. The method of claim 1, wherein the testosterone or derivative thereof is administered to the subject via intramuscular injection.

15. The method of claim 1, wherein the growth hormone or derivative thereof is administered at about 0.01 μg/kg/day to about 250 μg/kg/day/kg/day.

16. The method of claim 14, wherein the growth hormone or derivative thereof is administered at about 2.5 μg/kg/day to about 6.0 μg/kg/day or about 4.0 μg/kg/day to about 5.5 μg/kg/day.

17. (canceled)

18. The method of claim 16, wherein the growth hormone or derivative thereof is administered at about 5.0 μg/kg/day.

19. The method of claim 1, wherein the growth hormone or derivative thereof is administered to the subject via subcutaneous injection.

20. The method of claim 1, wherein the testosterone or derivative thereof, or the growth hormone or derivative thereof is administered for a duration of at least 1 week.

21. The method of claim 19, wherein the duration is 8 to 36 weeks, 12 to 30 weeks, or about 24 weeks.

22. (canceled)

23. (canceled)

24. A method for treating FSHD in a subject comprising:
administering to the subject an effective amount of testosterone or a derivative thereof, wherein the testosterone or derivative thereof is administered at about 0.1 mg to about 30,000 mg, and
administering to the subject an effective amount of growth hormone or a derivative thereof, wherein the growth hormone or derivative thereof is administered at about 0.01 μg/kg/day to about 250 μg/kg/day/kg/day.

25. A kit for (i) improving muscle mass, muscle strength, or muscle function, (ii) treating a disorder associated with muscle wasting or muscle weakness, or (iii) reducing fatigue, pain, or obesity in a subject in need thereof, the kit comprising:
(i) an effective amount of testosterone or a derivative thereof; and
(ii) an effective amount of growth hormone or a derivative thereof.
